(19) United States
(12) Patent Application Publication
(10) Pub. No.: US 2010/0249138 A1
(43) Pub. Date: Sep. 30, 2010

(54) MPZP: A SMALL MOLECULE CORTICOTROPIN-RELEASING FACTOR TYPE 1 RECEPTOR (CRF1) ANTOGONIST

(76) Inventors: George F. Koob, Rancho Santa Fe, CA (US); Eric P. Zorrilla, San Diego, CA (US); Barbara Mason, Rancho Santa Fe, CA (US); Kim Janda, La Jolla, CA (US); Peter Wirsching, Del Mar, CA (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 12/677,770

(22) PCT Filed: Sep. 12, 2008
(86) PCT No.: PCT/US08/76257

§ 371 Date: Mar. 11, 2010

Related U.S. Application Data
(60) Provisional application No. 60/972,409, filed on Sep. 14, 2007.

(51) Int. Cl. A61K 31/53; A61K 31/519

(52) U.S. Cl. 514/246; 514/264.11; 514/259.3

(57) ABSTRACT

A method for treating or preventing a host mammal that exhibits aversive signs and symptoms present during protracted abstinence or extended discontinuation syndromes as seen after cessation of compulsive activity, behaviors, or substance use is disclosed. That method comprises administering to a host mammal in need a pharmaceutical composition containing an aversive sign and symptom lessening amount a compound of Formula I or a pharmaceutically acceptable salt thereof dissolved or dispersed in a physiologically acceptable diluent, and repeating the administration as needed,

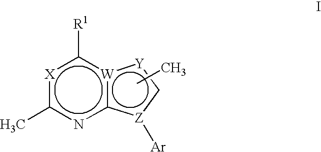

wherein W, X, Y and Z, R¹ and Ar are defined within. Data are provided in rats as host mammals using behavioral models dependent on the CRF₁ system: defensive burying, alcohol dependence, cocaine dependence and nicotine dependence. A contemplated method also is useful for inhibiting relapse of such a behavior. A contemplated method also is useful for treating substance-related or substance-induced psychiatric disorders that include aversive signs and symptoms.

### GOVERNMENTAL SUPPORT

The present invention was made with governmental support from National Institutes of Health grants AA06420, AA08459, and AA12602 from the National Institute on Alcohol Abuse and Alcoholism, National Institute on Drug Abuse Grant DA004398, and grant DK26741 from the National Institute of Diabetes and Digestive and Kidney Diseases. The government has certain rights in the invention.

### TECHNICAL FIELD

The present invention contemplates a method of modulating the behaviour of a host mammal that exhibits aversive signs and symptoms present during protracted abstinence or extended discontinuation syndromes following cessation of compulsive activity, behaviors, or substance use, as well as preventing the occurrence of such behavior. More particularly, the invention contemplates a method of modulating, modifying, or preventing such behavior by administering a small molecule CRF₁ antagonist to a host mammal in need.

### BACKGROUND ART

Corticotropin-releasing factor (CRF) is a 41 amino-acid residue peptide that mediates neuroendocrine [Vale et al., Science (1981) 213:1394-1397] and behavioral responses to stress [Sutton et al., Nature (1982) 297:331-333; Britton et al., Life Sci. (1986a) 39:1281-1286, Britton et al., Brain Res. (1986b) 369:303-306]. CRF and its putative receptors are now recognized to have numerous endogenous functions and are currently being explored as therapeutic targets for intervention in stress-related disorders such as anxiety and alcohol dependence [Koob, Alcohol Clin. Exp. Res. (2003) 27:232-243; Cowen et al., CNS Neurol. Disord. Drug Targets (2006) 5:233-239; Gehlert et al., J. Neurosci. (2007) 27:2718-2726; Heilig et al., Pharmacol. Ther. (2006) 111:855-876].

CRF exerts its actions via two known receptors: Type I (CRF₁) [Chang et al., Neuron (1993) 11:1187-1195; Chen et al., Proc. Natl. Acad. Sci. USA (1993) 90:8967-8971; Perrin et al., Endocrinology (1993) 133:3058-3061] and Type II (CRF₂) [Lovenberg et al., Proc. Natl. Acad. Sci. USA (1995) 92:836-840].

Both receptors belong to the B1 subgroup of G protein-coupled receptors linked to a number of intracellular signaling pathways, including ligand-dependent increase of intracellular cyclic adenosine monophosphate (cAMP) [Chen et al., Brain Res. (1986) 381:49-57; Giguere et al., Proc. Natl. Acad. Sci. USA (1982) 79:3466-3469]. CRF cell bodies, terminals, or CRF receptors are located in neuroendocrine structures, such as the paraventricular nucleus of the hypothalamus, median eminence, and anterior pituitary, as well as in extrahypothalamic brain regions of the “extended amygdala” that are important for behavioral responses to stress and addictive disorders [Bloom et al., Regul. Pept. (1982) 4:43-48; Swanson et al., Neuroendocrinology (1983) 36:165-186].

Genetic and pharmacological evidence implicates CRF₁ in mediating anxiety-related behaviors in animals [Timpl et al., Nat. Genet. (1998) 19:162-166; Smith et al., Neuron (1998) 20:1093-1102; Heinrichs et al., Regul. Pept. (1997) 71:15-21; Liebsch et al., Regul. Pept. (1995) 59:229-239; McElroy et al., Psychopharmacology (2002) 165:86-92; Zorrilla et al., Brain Res. (2002) 952:200-210, Zorrilla et al., Eur Neuropsychopharmacol. (2003) 13(Suppl. 4):s130-131; Zorrilla et al., Expert Opin. Investig. Drugs (2004) 13:799-828]. CRF₁ knockout mice display less anxiety-like behavior [Timpl et al., Nat. Genet. (1998) 19:162-166; Smith et al., Neuron (1998) 20:1093-1102]. Central administration of CRF mimics the behavioral responses to stress in rodents [Britton et al., Life Sci. (1986a) 39:1281-1286; Britton et al., Brain Res. (1986b) 369:303-306; Sutton et al., Nature (1982) 297:331-333; Dunn et al., Brain Res. Rev. (1990) 15:71-100], and CRF₁ antagonists have opposing effects [Zorrilla et al., Exp. Opin. Invest. Drugs (2004) 13:799-828]. CRF₂ receptors appear to be related to appetite regulation and possibly anxiolytic-like responses [for review, see Fekete et al., Front. Neuroendocrinol. (2007) 28:1-27].

Alcoholism is a chronically relapsing disorder characterized by cycles of repeated high alcohol intake and negative emotional consequences during withdrawal [Breese et al., Alcohol Clin. Exp. Res. (2005) 29:185-195; Koob, Alcohol Clin. Exp. Res. (2003) 27:232-243; Heilig et al., Pharmacol. Ther. (2006) 111:855-876]. Alcoholics are thought to drink alcohol initially for its euphorigenic effects, and subsequently to avoid or reduce the negative emotional state experienced in the absence of the drug or to self-medicate preexisting negative emotional states Koob, Alcohol Clin. Exp. Res. (2003) 27:232-243; Cappell et al., Drug Alcohol Depend. (1979) 4:15-31; Lowman et al., Addiction (1996) 91(Suppl.):s51-71]. CRF activation of CRF₁ receptors is hypothesized to play a significant role in the negative emotional state and alcohol-seeking behavior associated with withdrawal from chronic alcohol exposure in rats [Koob, Alcohol Clin. Exp. Res. (2003) 27:232-243; Menzaghi et al., J. Pharmacol. Exp. Ther. (1994) 269:564-572; Valdez et al., Pharmacol. Biochem. Behay. (2004) 79:671-869]. Indeed, CRF₁ antagonists attenuate the elevated anxiety-like behavior [Overstreet et al., Pharmacol. Biochem. Behay. (2004) 77:405-413] and increased drinking [Chu et al., Pharmacol. Biochem. Behay. (2007) 86:813-821; Funk et al., Biol. Psychiatry (2007) 61:78-86; Gehlert et al., J. Neurosci. (2007) 27:2718-2726; Sabino et al., Psychopharmacology (2006) 189:175-186] associated with withdrawal in dependent animals.

Compounds that modulate the CRF₁ system are being developed for the treatment of alcohol dependence. Although peptide CRF₁ antagonists are available, they are not able to penetrate the blood-brain barrier, thereby limiting their clinical effectiveness for treating central nervous system (CNS) disorders. Alternatively, small molecule, non-peptide CRF₁ selective antagonists with appropriate physiochemical properties can readily reach the brain CRF system, and considerable effort is being made to develop and characterize such compounds [Zorrilla et al., Exp. Opin. Invest. Drugs (2004) 13:799-828; Kehne et al., Curr. Drug Targets CNS Neurol. Disord. (2002) 1:467-493].

Illustrative compounds that interact with the CRF₁ system include WO 2007039264, WO 2006044958, WO 20050799868, WO 2005063755, WO 2005051954, WO 2005113375, WO 2004058767, WO 2006102194, WO 2004088708, WO 2004037822, and WO 2004058767. Particular attention is drawn to WO 9938868 (U.S. Pat. No. 6,313,124; U.S. Pat. No. 6,191,131; and U.S. Pat. No. 6,060,478) and WO 9803510 (U.S. Pat. No. 6,124,289). It is believed that the functional activities of the compounds disclosed in the above documents are directed to short term effects, such as to treat acute detoxification syndromes, as compared to the longer term efficacy, such as to treat persistent and lasting consequences of compulsive use and behavioral disorders, that are disclosed hereinafter.

Tobacco addiction is the leading avoidable cause of disease and premature death in the U.S., responsible for over 400 000 deaths annually [Fellows et al., Morb Mort Rep (2002) 51:300-303; Henningfield et al., Ann NY Acad Sci (2000) 909:247-256]. The main psychoactive ingredient responsible for tobacco addiction has long been hypothesized to be nicotine.

Nicotine acutely produces modest positive reinforcing effects [Pomerleau et al., Psychopharmacology (Berl). (1992) 108: 460-465; Grunberg, N. E., Addiction (1994) 89: 1443-1446] by activating reward systems, including the mesolimbic dopamine system [Mansvelder et al., Neuron. (2002) 33:905-919; Nestler, Nature Neuroscience (2005) 8:1445-1449]. However, the transition from nicotine use to nicotine dependence has been hypothesized to result from neuroadaptative changes in the brain that produce a powerful need to continue tobacco use [Koob et al., Nature Neuroscience (2005) 8:1442-1444; Tiffany et al., Addiction (2004) 99: 78-86]. Such neuroadaptation may involve the mechanisms responsible for the negative emotional states observed during abstinence from nicotine in dependent individuals [Epping-Jordan et al., Nature. (1998) 393:76-79; Hughes et al., Addiction (1994) 89:1461-1470]. The negative emotional state produced by nicotine withdrawal is hypothesized to represent a powerful source of negative reinforcement leading to excessive drug intake.

Spontaneous and precipitated (using nicotinic receptor antagonists such as mecamylamine) nicotine withdrawal dramatically decreases brain reward function and the efficacy of natural reinforcers in rodents [Epping-Jordan et al., Nature. (1998) 393:76-79; Hughes et al., Addiction (1994) 89:1461-1470]. These effects occur despite the initial, weak reinforcing effect of nicotine suggesting there must be other mechanisms driving the development of nicotine dependence.

Nicotine, the main psychoactive ingredient of tobacco, induces negative emotional symptoms during abstinence that contribute to a profound craving for nicotine. However, the neurobiological mechanisms underlying how nicotine produces dependence remains poorly understood. Again, one mechanism for both the anxiety-like symptoms of withdrawal and excessive nicotine intake observed after abstinence, is via recruitment of the extrahypothalamic stress peptide corticotropin-releasing factor (CRF) system and activation of CRF₁ receptors. Overactivation of the CRF-CRF₁ system may contribute to nicotine dependence, and may represent a prominent target for investigating the vulnerability to tobacco addiction.

The development of drug dependence has been hypothesized to involve allostatic dysregulation of brain reward function [Koob et al., Science (1997) 278:52-58; Koob et al., Nat. Neurosci. (2005) 8:1442-1444]. In addition to disruption of reward neurotransmission, the recruitment of brain stress systems, such as noradrenergic and CRF neurocircuitry, may contribute to the onset of drug dependence and addiction [Koob, Eur. Neuropsychopharmacol (2003) 13:442-452]. Dysphoria and anxiety are reported among the major drug withdrawal symptoms in drug-dependent humans [Kampman et al., Addict. Behay. (1998) 23:449-461], and CRF and norepinephrine may possess a feed-forward interaction in driving anxiety-like responses [Koob, Biol. Psychiatry (1999) 46:1167-1180).

The hypothesis that CRF-CRF₁ systems may be involved in withdrawal-induced anxiety-like responses is supported by research showing an association between cocaine withdrawal and increased extracellular CRF levels in the amygdala, as well as depleted amygdala CRF tissue content [Richter et al., Synapse (1999) 32:254-261; Zorrilla et al., Psychopharmacology (2001) 158: 374-381]. Intracranial administration of a peptide CRF receptor antagonist blocks cocaine withdrawal-induced anxiety-like behavior in rats [Basso et al., Psychopharmacology (1999) 145:21-30]. An interplay between cocaine and the brain stress systems is demonstrated by the facilitation of cocaine self-administration in rats that are high-responding to novelty [Piazza et al., J. Neurosci. (2006) 20:4226-4232] and the blockade of acquisition and maintenance of cocaine self-administration following bilateral adrenalectomy [Goeders et al., Brain Res. (1996) 722:145-152].

Additionally, a specific stressor, such as noncontingent footshock stress, increases corticosterone and is correlated with an increased acquisition rate of cocaine self-administration [Goeders et al., Psychopharmacology (1994) 114:63-70; Goeders et al., Neuroendocrinology (1996) 64:337-348]. Similarly, repeated corticosterone treatments promote the acquisition of cocaine self-administration [Mantsch et al., J. Pharmacol. Exp. Ther. (1998) 287:72-80]. There also is downregulation of CRF receptors in the medial prefrontal cortex, nucleus accumbens, olfactory tubercle, and amygdala following cocaine administration [Goeders et al., Brain Res, (1990) 531:322-328]. Pretreatment with the CRF₁ antagonist CP-154,526 results in dose-dependent decreases in cocaine self-administration in rats with limited daily drug access (1 h/day), yet does not affect food-reinforced responding [Goeders et al., Neuropsychopharmacology (2000) 23:577-586]. CRF system involvement is not limited to the acquisition and maintenance of cocaine self-administration. It was shown that CRF system stimulation induces reinstatement of cocaine-seeking behavior in rats during withdrawal [Erb et al., Psychopharmacology (2001) 158:360-365; Erb et al., Psychopharmacology (2006) 187:112-120].

Taken together, these data suggest that activation of CRF systems in the brain may be involved in the development of the emotional dysregulation hypothesized to motivate intake in ethanol or drug dependence, and, more generally, maintenance or resumption of compulsive activity in other compulsive behavioral disorders.

In the published reports of some of the inventors and their co-workers, daily, extended access to cocaine has been shown to produce an increase in self-administration of the drug over sessions (termed “escalation”) [Ahmed et al., et al. Science (1998) 282:298-300; Wee et al. (2007) J Pharmacol Exp Ther (2007) 320:1134-1143]. The increased drug intake with extended access in rats provides some face validity for compulsive drug intake in humans [American Psychiatric Association, Diagnostic and Statistical Manual of Mental Disorders, 4th edition, American Psychiatric Press, Washington D.C. (2000)]. Furthermore, the increased cocaine self-administration with extended access strongly correlates with an increasing threshold for intracranial self-stimulation [Ahmed et al., Nat. Neurosci. (2002) 5:625-626], suggesting decreased brain reward function during the escalation of cocaine self-administration. This finding, in turn, supports the construct validity of this model for the development of drug dependence [Koob et al., Pharmacol. Biochem. Behay. (1997) 57:513-521; Koob et al., Science (1997) 278:52-58; Sinha et al., Psychopharmacology (2000) 152:140-148].

Most of the presently available non-peptide CRF₁ antagonists are more lipophilic than prototypical CNS therapeutics [(Zorrilla et al., Exp. Opin. Invest. Drugs (2004) 13:799-828]. Contrarily, the present invention utilizes the pharmacological and behavioral properties of a non-peptide small molecule CRF₁ specific antagonist compound with hydrophilicity approaching that of typical CNS therapeutics.

### BRIEF SUMMARY OF THE INVENTION

The present invention contemplates a method of treatment using a compound corresponding in structure to Formula I and the pharmaceutically acceptable acid addition salts thereof,

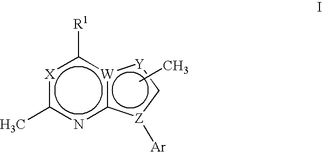

wherein

W and Z are independently N or C, and X and Y are independently N or CH, with the proviso that at least two and no more that three of W, X, Y and Z are N;

R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;

Ar— is

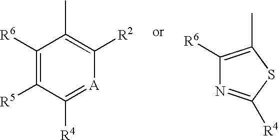

wherein

A is CH or N,

R² is selected from the group consisting of hydrido, methyl, methoxy, chloro and bromo,

R⁴ is selected from the group consisting of chloro, methyl, methoxy, dimethylamino and morpholinyl,

R⁵ is selected from the group consisting of hydrido, chloro and methyl, and

R⁶ is selected from the group consisting of hydrido, chloro, methyl and methoxy. A contemplated compound exhibits a calculated cLogD, pH 7 value of about 1.5 to about 4.5, using ACD/Labs Software v.8.14 for Solaris, a pKₐ value of about 4 to about 8.5, and a polar surface area of about 40 to about 70 Å². In accordance with a contemplated method, a mammalian host animal in need thereof is administered a pharmaceutical composition

A method for treating a host mammal that exhibits aversive signs and symptoms present during protracted abstinence or extended discontinuation syndromes as seen after cessation of compulsive activity, behaviors, or substance use is contemplated. Illustrative protracted abstinence or extended discontinuation syndromes include those that result from cessation of use of alcohol, licit or illicit drugs, or preferred foods, as well as from compulsive behaviors, such as shopping, gambling, sex, or computer use. That method comprises administering a pharmaceutical composition containing an aversive sign and symptom lessening amount of a compound of Formula I or its pharmaceutically acceptable acid addition salt dissolved or dispersed in a physiologically acceptable diluent to a host mammal in need thereof, and repeating the administration as needed.

A method is also contemplated for treating substance-related or substance-induced psychiatric disorders that include aversive signs and symptoms. This method comprises administering a pharmaceutical composition containing a substance-related or substance-induced psychiatric disorder aversive sign and symptom lessening amount of a compound of Formula I or its pharmaceutically acceptable acid addition salt dissolved or dispersed in a physiologically acceptable diluent to a host mammal in need thereof, and repeating the administration as needed.

A method for inhibiting relapse to the above recited compulsive use or behavioral disorders is further contemplated. Here, the utility also involves the treatment of subjective craving symptoms, which can include symptoms of anxiety, dysphoria, tension, irritability, and depressed mood, in addition to behavioral measures of relapse. In this method, a pharmaceutical composition containing a compulsive use or behavioral disorders relapse inhibiting amount of a compound of Formula I or its pharmaceutically acceptable acid addition salt dissolved or dispersed in a physiologically acceptable diluent is administered to a host mammal in need thereof, and repeating the administration as needed.

A method for preventing a host mammal from exhibiting aversive signs and symptoms present during protracted abstinence or extended discontinuation syndromes as seen after cessation of compulsive activity, behaviors, or substance use is still further contemplated. In this method, a pharmaceutical composition containing an aversive sign and symptom present during protracted abstinence or extended discontinuation syndrome-preventing amount of a compound of Formula I or its pharmaceutically acceptable acid addition salt dissolved or dispersed in a physiologically acceptable diluent is administered to a host mammal in need thereof, and repeating the administration as needed.

A particularly preferred compound of Formula I, named N,N-bis(2-methoxyethyl)-3-(4-methoxy-2-methylphenyl)-2,5-dimethyl-pyrazolo[1,5-a]pyrimidin-7-amine and referred to herein as MPZP, is depicted below.

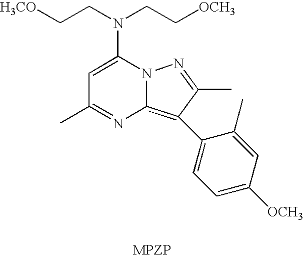

Carrying out a contemplated method in a preclinical test setting has been performed using MPZP an exemplar compound of Formula I. These studies have shown that MPZP reduces the elevated levels of alcohol or drug (such as nicotine, heroin, cocaine) self-administration associated with abstinence from use in animals with an extensive history of previous exposure to alcohol or to the drug of abuse. MPZP also reduced the anxiety-like state associated with abstinence from the drug of abuse in animal models.

Also contemplated herein is the use of a compound corresponding in structure to Formula I and the pharmaceutically acceptable acid addition salts thereof in the preparation of a medicament for treating a host mammal that exhibits aversive signs and symptoms present during protracted abstinence or extended discontinuation syndromes as seen after cessation of compulsive activity, as well as for the preparation of a medicament for treating substance-related or substance-induced psychiatric disorders that include aversive signs and symptoms, a medicament for inhibiting relapse to the above compulsive use or behavioral disorders, and a medicament for preventing a host mammal from exhibiting aversive signs and symptoms present during protracted abstinence or extended discontinuation syndromes as seen after cessation of compulsive activity, behaviors, or substance use.

### BRIEF DESCRIPTION OF THE DRAWINGS

In the drawings that form a portion of the specification:

FIG. 1 is a graph that illustrates the binding affinity of MPZP and DMP904 for CRF receptors in rat cerebellar homogenates by showing the displacement of specific [¹²⁵I]-Tyr⁰-sauvagine binding from rat cerebellar membrane homogenates by unlabeled MPZP or the reference CRF₁ antagonist DMP904. Data points represent mean inhibition observed across six independent experiments. Curves were fit using a four-parameter, single-site logistic regression equation.

FIG. 2 is a photograph of an autoradiograph of CRF receptors in rat brain in which slide-mounted coronal rat brain sections (20 μm) were incubated with [¹²⁵I]Tyr⁰-sauvagine (0.2 nM) at the level of the lateral septum (left panels, FIGS. 2-A, -C, -E and -G) or ventromedial nucleus of the hypothalamus (right panels, FIGS. 2-B, -D, -F, and -H.). Representative autoradiographic images are shown from sections that were co-incubated with assay buffer only (panels 2-A, 2-B; “total binding”); with the high affinity selective CRF₁ antagonist R121919 (1 μM) to displace specific radioligand binding from CRF₁ receptors (2-C, 2-D; “non-CRF₁ binding”); with the putative, selective CRF₁ antagonist under study, MPZP (3 μM) (2-E, 2-F); or with the subtype non-selective CRF₁/CRF₂ antagonist D-Phe-CRF₁₂₋₄₁ (300 nM) to displace specific radiolabel binding from CRF₁ and CRF₂ receptors (2-G, 2-H; “non-CRF₁/CRF₂ binding”). Backgrounds were subtracted from all images using ImageJ (National Institutes of Health, Bethesda, Md.).

FIG. 3 shows two graphs of the anxiolytic-like effect of MPZP on the defensive burying model of active anxiety-like behavior [FIG. 3-A: latency to bury; FIG. 3-B (inset line graph): burying duration (s) across time (2.5 minute bins); B (bar graph): total burying duration (s)]. MPZP increased the latency to first engage in burying behavior following contact with the shock probe (A). MPZP reduced defensive burying time (B, inset line graph and bar graph), and the attenuating effects of MPZP on burying time did not significantly differ across 2.5 minute bins. *All Ps<0.05 compared to vehicle (0 mg/kg MPZP) treated controls. Data are shown as mean±SEM. (n=6-11 rats per dose).

FIG. 4 contains three graphs that relate to operant self-administration behavior prior to and following dependence induction via chronic intermittent alcohol vapor exposure (gray shading). Post-vapor testing was conducted when dependent animals were in acute withdrawal (6-8 hours after removal from vapors). There were main effects of Vapor treatment (dependent vs. nondependent animals) and Test session (pre- vs. post-vapor tests) and an interaction between these two factors on alcohol self-administration (g/kg intake, A; lever presses/responses, B). Post hoc analyses indicated that post-vapor alcohol self-administration in dependent animals was higher than post-vapor alcohol self-administration in nondependent animals (*, A, B) and compared to pre-vapor alcohol self-administration (#, A, B). There was a main effect of Test session on water self-administration such that post-vapor water self-administration was slightly, but significantly, lower on the post-vapor test 2 compared to pre-vapor water self-administration tests (#, C). However, there were no differences in water self-administration either before or after vapors in dependent animals compared to nondependent controls. *Compared to nondependent controls. # Compared to pre-vapor test sessions (all Ps<0.05). Data are shown as mean±SEM (n=8 per vapor treatment group).

FIG. 5 contains two bar graphs (FIGS. 5A and 5B) whose data illustrate the effect of MPZP on operant self-administration of alcohol (g/kg) and water (responses) in dependent and nondependent rats. Testing was conducted when dependent animals were in acute withdrawal (6-8 hours after removal from vapors). There were main effects of Vapor treatment (dependent vs. nondependent animals) and MPZP dose (0, 5, 10, 20 mg/kg) on alcohol self-administration (g/kg intake) detected using ANOVA and dose-response fit analyses. Overall, dependent animals self-administered significantly more alcohol than nondependent animals (*, FIG. 5A). MPZP significantly reduced alcohol self-administration only in dependent animals, indicated by a significant downward sigmoidal trend (r²=0.907, P<0.05; ED₅₀=10.68 mg/kg MPZP, no indicator) and a reduction with the 20 mg/kg dose compared to vehicle (0 mg/kg MPZP) (#, FIG. 5A). MPZP had no effect on alcohol self-administration in nondependent animals (FIG. 5A) or on water self-administration (responses) in either dependent or nondependent animals (FIG. 5B). Note: Alcohol self-administration data are expressed in g/kg intake, a more pharmacologically informative measure of alcohol consumption than lever responses, but the pattern of changes seen for alcohol responses was similar to that for g/kg (dependent animals: 0 mg/kg=89±11, 5 mg/kg=72±14, 10 mg/kg=70±7, 20 mg/kg=47±7; nondependent animals: 0 mg/kg=32±4, 5 mg/kg=37±5, 10 mg/kg=32±5, 20 mg/kg =33±4). *Compared to nondependent controls. #Compared to vehicle (0 mg/kg MPZP) (all Ps<0.05). Data are shown as mean ±SEM (n=8 per vapor treatment group; MPZP doses were administered using a within-subjects Latin square design).

FIG. 6 is s series of three graphs (FIGS. 6A-6C) that illustrate the effects of mecamylamine-precipitated nicotine withdrawal on extracellular levels of CRF-like-immunoreactivity (CRF-L-IR) in the central nucleus of the amygdala and CRF antagonist blockade of precipitated withdrawal induced anxiety-like behavior in rats using the defensive burying test. FIG. 6A shows the effect of mecamylamine (1.5 mg/kg, i.p.) -precipitated withdrawal on extracellular levels of CRF-L-IR in the central nucleus of the amygdala as measured by in vivo microdialysis in chronic nicotine pump-treated (nicotine dependent, n=7) and chronic saline pump-treated (non-dependent, n=6) rats (*p<0.05 vs. non-dependent). FIG. 6B shows CRF-L-IR levels expressed as percentage of baseline (first 3 samples) during the first four samples after vehicle or mecamylamine injections (*p<0.05 vs. vehicle). FIG. 6C shows CRF₁ antagonist blockade of precipitated-withdrawal induced anxiety-like behavior in rats using the defensive burying test. Mecamylamine (1.5 mg/kg, i.p.) injection in nicotine-dependent rats increased the time spent burying (*p<0.05 vs. vehicle), an effect blocked by pretreatment with the CRF₁ antagonist (MPZP, 4 mg/kg s.c., −1 hour) (n=7-9 per group, ^{#}p<0.05 vs. mecamylamine). Data represent mean±SEM.

FIG. 7 contains five graphs (FIGS. 7A-7E) that show characterization of the nicotine deprivation effect. FIG. 7A shows total (23 hour) active and inactive responses after repeated cycles of 72 hours of nicotine deprivation (ND), followed by 4 days of self-administration (*p<0.05 vs. baseline). FIG. 7B is a scatter plot of nicotine intakes observed during the first session before (pre-ND) and after (post-ND) each of the four cycles of nicotine deprivation that shows the robustness of the nicotine deprivation effect. The numbers represent the percentage of measures above and below the y=x line. FIG. 7C shows the reliability of the nicotine deprivation effect via correlation of post-ND nicotine intakes between each of the four cycles (ND₍₋₁₎ vs. ND₍₀₎=ND₁ vs. ND₂, ND₂ vs. ND₃, ND₃ vs. ND₄). FIG. 7D illustrates the coefficient of variation of post-ND intakes between subjects vs. within subjects (*p<0.05). FIG. 7E shows the effect of duration of abstinence (hours) on active responses during the subsequent 12 hour period of nicotine access. (*p<0.05 vs. 1 hour). Note logarithmic time scale. Dotted lines represent mean±sem of the 1 hour time point. (*p<0.05 vs. 1 h). Data represent mean±SEM.

FIG. 8 in two panels (FIGS. 8A and 8B) illustrate the specificity of the nicotine deprivation effect. FIGS. 8A and 8B show total responses during the entire session in rats given extended (23 hours, n=7) (FIG. 8A), or limited (1 hour, n=6) (FIG. 8B) access to nicotine before and after 72 hours of abstinence. (*p<0.05 vs. baseline). Data represent mean±SEM.

FIG. 9 in three panels (FIG. 9A-9C) illustrates that abstinence-induced escalation of nicotine intake is blocked by a CRF₁ receptor antagonist. Thus, FIG. 9A shows the effect of a CRF₁ antagonist (MPZP, s.c., −1 hour) on nicotine self-administration during the active period in rats given extended access to nicotine (*p<0.05 vs. baseline, ^{#}p<0.05 vs. post abstinence vehicle treatment, n=8).

FIG. 9B shows the correlation between magnitude of the nicotine deprivation effect and percentage changes in the nicotine deprivation effect after CRF₁ antagonist, in which the higher the nicotine deprivation effect, the more effectively the antagonist blocked self-administration of nicotine (r=−0.71 p<0.05). The x-axis represents active responses after vehicle injection, whereas the y-axis represents the reduction in active responses after the highest dose of MPZP (20 mg/kg), in percentage changes compared with active responses after vehicle injection. FIG. 9C shows a lack of effect of the CRF₁ receptor antagonist (MPZP, s.c., −1 hour) on baseline nicotine self-administration responding in rats given limited access to nicotine (n=10). Data represent mean±SEM.

FIG. 10 in two panels (FIGS. 10A and 10B) illustrate self-administration of cocaine during the escalation period of testing during the total session (FIG. 10A) or first hour of intake (FIG. 10B) under a fixed-ratio 1 schedule of reinforcement. The data represent mean (+SEM) cocaine intake adjusted for body weight (mg/kg). Open symbols are the data for rats during the first hour of sessions (ShA, n=16, LgA, n=16). Filled symbols are the data for rats during the total 6 h session (LgA). *p<0.05, **p<0.01, ***p<0.001 compared to session 1 (Student-Newman-Keuls test).

FIG. 11 contains a series of bar graphs that show antalarmin effects on cocaine intake in ShA and LgA rats. There was a main effect for Access (F_{1,15}=5.446, *p<0.05) and a significant Access×Antalarmin Dose interaction based on a linear trend contrast analysis (F_{1,15}=5.05, +p<0.05). The CRF₁ antagonist antalarmin significantly decreased cocaine self-administration in LgA (n=9) rats at the highest dose (25 mg/kg) (‡p<0.05, Dunnett's test) without significantly altering cocaine self-administration in the ShA group (n=8). Test sessions lasted 1 hour. Data are expressed as mean (+SEM) cocaine intake (mg/kg).

FIG. 12 contains bar graphs that show MPZP effects on cocaine intake in ShA and LgA rats under a fixed-ratio schedule. MPZP was subcutaneously injected 45 minutes before a test session. Test sessions lasted 1 hour and were separated by one or two treatment-free escalation sessions. MPZP decreased cocaine intake in both ShA and LgA rats. Two-way ANOVA revealed an overall main effect for access (F1, 13=7.249, p<0.05), a main effect for MPZP dose (F3, 39=10.076, p<0.001), but no interaction. However, a two-way linear contrast showed a significant dose x access linear contrast interaction (F1,13=5.48, p<0.05). Additionally, there was a simple main effect of dose on cocaine intake both in ShA and LgA rats (LgA, F3,39=9.31, p<0.01; ShA, F3,39=4.08, p<0.05). Post hoc Dunnett's test showed a significant decrease of cocaine intake at 10 mg/kg (p<0.05) and 27.5 mg/kg (p<0.01) in LgA rats and at 27.5 mg/kg in ShA rats (p<0.05). Data are expressed as mean (+SEM) cocaine intake (mg/kg). *p<0.05, **p<0.01 compared with the vehicle.

The present invention has several benefits and advantages. One benefit is that its use is effective in treating aversive symptoms and signs that are long term, as compared to treatment of the more usually treated, short term aspects of dependencies, such as acute withdrawal, which emerges quickly during detoxification and lessens thereafter.

An advantage of the invention is that its treatment method can also be used as a preventative or inhibitory therapy for relapse to the previous addictive behavior.

Still further benefits and advantages will be apparent to the worker of ordinary skill from the discussion that follows.

### DETAILED DESCRIPTION OF THE INVENTION

The present invention contemplates a method of treatment using a compound corresponding in structure to Formula I and the pharmaceutically acceptable acid addition salts thereof,

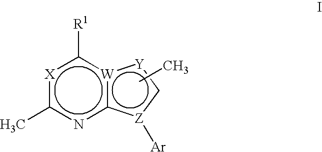

wherein

W and Z are independently N or C, and X and Y are independently N or CH, with the proviso that at least two and no more that three of W, X, Y and Z are N;

R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;

Ar— is

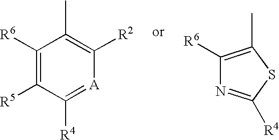

wherein

A is CH or N,

R² is selected from the group consisting of hydrido, methyl, methoxy, chloro and bromo,

R⁴ is selected from the group consisting of chloro, methyl, methoxy, dimethylamino and morpholinyl,

R⁵ is selected from the group consisting of hydrido, chloro and methyl, and

R⁶ is selected from the group consisting of hydrido, chloro, methyl and methoxy.

A contemplated compound exhibits a calculated cLogD, pH 7 value of about 1.5 to about 4.5, and preferably about 2.0 to about 3.5 using ACD/Labs Software v.8.14 for Solaris. More preferably, the calculated cLogD, pH 7 value is about 2.5 to about 3.0. A contemplated compound has a pKₐ value of about 4 to about 8.5, and more preferably about 5.0 to about 7.5. A contemplated compound has a calculated polar surface area of about 40 to about 70 Å², and preferably about 45 to about 60 Å².

A contemplated compound can be looked at for ease of discussion as containing three portions, a fused 6/5-membered ring core that is bonded to an R¹ substituent group and to an Ar substituent group. Each of those three component portions will be discussed below for convenience as will illustrative completed compounds.

Looking first at the core, one sees that it is aromatic, is comprised of a 6-membered ring fused to a 5-membered ring. The core contains 3 or 4 nitrogen atoms in the rings, of which one is depicted as present in the same position in the 6-membered ring in each molecule (the constant nitrogen atom), whereas the other two or three nitrogen atoms are in variable ring positions that are depicted by the letters W, X, Y and Z. The core also contains a methyl substituent bonded adjacent to the constant nitrogen atom of the 6-membered ring and at one of two positions in the 5-membered ring.

The core in generic form corresponds to structural Formula II with bond lines for substituents R¹ and Ar.

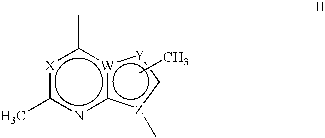

Specific, illustrative core structures are shown below as Formulas IIA-IID

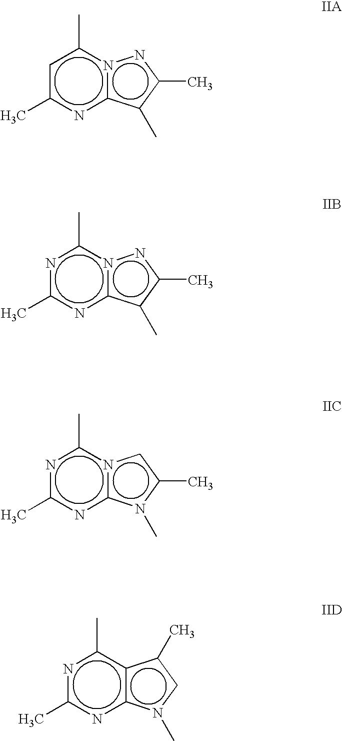

A core corresponding in structure to Formula IIA is particularly preferred.

A contemplated R¹ group is a disubstituted amine that contains two to about ten carbon atoms in the substituents. More specifically, R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₄ alkyl or C₁-C₄ alkenyl, mono-or dihydroxy-C₁-C₄ alkyl or C₁-C₄ alkenyl, N-methylamino-C₁-C₄ alkyl or C₁-C₄ alkenyl, 2- or 3-tetrahydrofuryl, and 2- or 3-tetrahydrofurfuryl. It is preferred that both of R⁷ and R⁸ be the same substituent group. It is also preferred that the R⁷ and R⁸ groups each contain a methoxy or hydroxy group. Thus, illustrative individual R⁷ and R⁸ substituent groups include methyl, ethyl, isopropyl, propyl, n-butyl, sec-butyl, t-butyl, hydroxymethyl, 2-hydroxypropyl, 3-hydroxypropyl, 2,3-dihydroxy-propyl, 3,4-dihydroxybutyl, 2-hydroxy-3-methoxypropyl, vinyl, allyl, 2-butenyl, methoxymethyl, methyoxyethyl, 2-methoxypropyl, 3-methoxypropyl, 4-methoxybutyl, 2-(methylamino)-propyl, 3-(methylamino)propyl, 4-(methylaminobutyl), 2-tetrahydrofuryl, 3-tetrahydrofuryl, 2-tetrahydrofurfuryl, 3-tetrahydrofurfuryl and the like. A particularly preferred R¹ group is a bis(2-methoxyethyl)amino group.

In an alternative embodiment, the substituent NR⁷R⁸ together forms a 5- or 6-membered ring containing zero or one oxygen atom in the ring, which ring is unsubstituted or substituted with a hydroxyl group, a hydroxymethyl group or a hydroxyethyl group. Thus, illustrative NR⁷R⁸ ring groups include N-piperidyl, N-(4-hydroxymethyl)-piperidyl, N-(3-hydroxymethyl)piperidyl, N-(4-hydroxyethyl)piperidyl, N-morpholinyl, N-(3-hydroxymethyl)morpholinyl, and the like.

Of the before-mentioned NR⁷R⁸ groups, those containing one or two oxygen atoms are preferred. Of the oxygen atom-containing substituents, those containing one or two methoxy groups or one or two hydroxy groups are more preferred.

As noted above, an Ar substituent can have one of two general formulas, IIIA and IIIB, that are shown below:

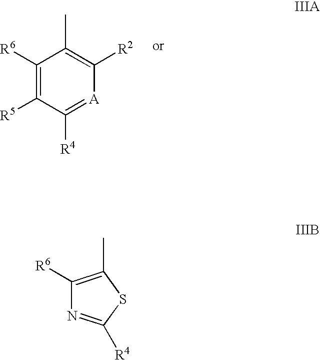

where “A” is N or CH. Thus, where A is N, the substituent IIIA is a substituted 3-pyridyl group having the structure of Formula IIIA-1, whereas where A is CH, the substituent IIIA is a substituted phenyl group whose structure is shown in Formula IIIA-2.

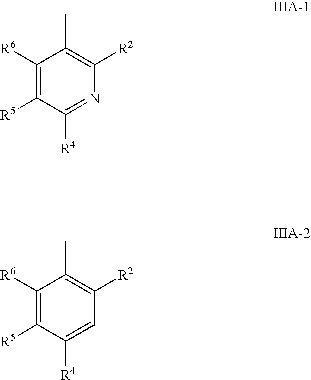

In a preferred embodiment, Ar conforms to structural Formula IIIA-2, wherein R² is methyl, R⁴ is methoxy and each of R⁵ and R⁶ is hydrido (H).

A particularly preferred compound of Formula I thus includes a N,N-bis-(2-methoxyethyl)-amino R¹ substituent linked to a core corresponding in structure to Formula IIA, that is linked to an Ar substituent that is a 2-methyl-4-methoxyphenyl group (Formula IIIA in which A=CH, R²=methyl and R⁴=methoxy). A particularly preferred compound so described can be named N,N-bis(2-methoxyethyl)-3-(4-methoxy-2-methylphenyl)-2,5-dimethyl-pyrazolo[1,5-a]pyrimidin-7-amine and referred to herein for brevity as MPZP, is depicted below, and is used illustratively herein as a contemplated compound in treatment methods as are described hereinafter.

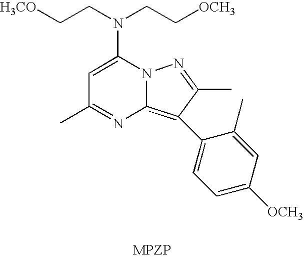

### Compositions and Methods

A contemplated compound can be used as the compound itself, but is typically present and used in the form of a pharmaceutically acceptable salt. The terms “pharmaceutically acceptable” and “physiologically acceptable” are used adjectivally herein to mean that the modified noun is appropriate for use in a pharmaceutical product.

Many exemplary pharmaceutically acceptable acids that are useful herein and whose salts with pharmaceutical compounds are used through out the world in approved products are disclosed in Berge, “Pharmaceutical Salts,” J. Pharm. Sci., 66(1):1-19 (January 1977). These salts include without limitation hydrochloric acid, hydrobromic acid, phosphoric acid, sulfuric acid, phosphoric acid, methanesulfonic acid, benzene sulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, benzenesulfonic acid and the like. As such, a contemplated compound is often present in the form of an amine salt derived from an inorganic or organic acid. Exemplary acid salts using some of the above acids include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate, benzenesulfonate (besylate) and undecanoate.

A pharmaceutical composition containing an effective amount of a compound of Formula I or its pharmaceutically acceptable salt dissolved or dispersed in a physiologically acceptable diluent is also contemplated. Total daily dose administered to a host mammal in need of treatment or prevention of aversive signs and symptoms present during protracted abstinence or extended discontinuation syndromes in single or divided doses of a compound of Formula I or its pharmaceutically acceptable salt in an amount effective to lessen or to prevent an aversive sign and symptom; i.e., an aversive sign and symptom lessening amount.

As is the case with most pharmaceutical products, an aversive sign and symptom lessening amount can vary with the particular pharmaceutical compound or its pharmaceutically acceptable salt, the host mammal in need of medication, as well as the age, weight and sex of the host mammal. An illustrative effective amount is about 0.01 to about 100 mg/kg body weight daily, preferably about 0.1 to about 50 mg/kg body weight daily, and more usually about 1 to about 30 mg. Dosage unit compositions can contain such amounts or submultiples thereof to make up the daily dose. A suitable dose can be administered, in multiple sub-doses per day. Multiple doses per day can also increase the total daily dose, should such dosing be desired by the person prescribing the drug.

A compound or its pharmaceutically acceptable salt useful in the present invention is typically formulated as a pharmaceutical composition that contains a compound of Formula I or a pharmaceutically acceptable salt thereof dissolved or dispersed in a physiologically acceptable diluent. Such a composition can then be administered orally, parenterally, by inhalation spray, rectally, or topically in dosage unit formulations containing conventional nontoxic pharmaceutically acceptable carriers, adjuvants, and vehicles as desired. Topical administration can also involve the use of transdermal administration such as transdermal patches or iontophoresis devices. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Suppositories for rectal administration of the drug can be prepared by mixing the drug with a suitable nonirritating excipient such as cocoa butter, synthetic mono-, di- or triglycerides, fatty acids and polyethylene glycols that are sold at ordinary temperatures but liquid at the rectal temperature and will therefore melt in the rectum and release the drug.

Solid dosage forms for oral administration can include capsules, tablets, pills, powders, and granules. In such solid dosage forms, the compounds of this invention are ordinarily combined with one or more adjuvants, excipients or other diluents appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions that comprise the physiologically acceptable diluent. These solutions and suspensions can be prepared from sterile powders or granules having one or more of the carriers or diluents mentioned for use in the formulations for oral administration. The compounds can be dissolved or dispersed in water, polyethylene glycol, propylene glycol, ethanol, corn oil, cottonseed oil, peanut oil, sesame oil, benzyl alcohol, sodium chloride, and/or various buffers. Other adjuvants, excipients or other diluents and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

A method for treating aversive signs and symptoms present during protracted abstinence or extended discontinuation syndromes that result from compulsive activity is particularly contemplated. That method comprises administering a composition containing an aversive sign and symptom lessening amount of a compound of Formula I or its pharmaceutically acceptable acid addition salt dissolved or dispersed in a physiologically acceptable diluent to a mammal in need thereof, and repeating the administration as needed. Illustrative protracted abstinence or extended discontinuation syndromes include those that result from protracted abstinence from use of alcohol, licit or illicit drugs, or preferred foods, as well as from compulsive behaviors, such as shopping, gambling, sex, or computer use.

Those familiar with the state of the art will recognize that protracted abstinence and extended discontinuation syndromes are distinct from withdrawal, which emerges quickly during detoxification, for example after only a few hours or days of cessation of use or of the behavior (e.g., DSM-IV Substance Withdrawal Delirium, “develops over a short period of time [usually hours to days]. . . . ” 291.0 Alcohol; 292.81 Sedative, Hypnotic, or Anxiolytic; 292.81 Other [or Unknown] Substance). Protracted abstinence involves a later emerging and longer lasting set of signs and symptoms.

Contemplated substance-related or substance-induced psychiatric disorders are exemplified in the Diagnosis and Statistical Manual Version IV, published by the American Psychiatric Association (1995) (hereinafter, DSM-IV). Such disorders can include substance-induced anxiety disorders (e.g., (291.8 (new code as of Oct. 1, 1996: 291.89) Alcohol; 292.89 Amphetamine (or Amphetamine-Like Substance); 292.89 Caffeine; 292.89 Cannabis; 292.89 Cocaine; 292.89 Hallucinogen; 292.89 Inhalant; 292.89 Phencyclidine (or Phencyclidine-Like Substance); 292.89 Sedative, Hypnotic, or Anxiolytic; 292.89 Other [or Unknown] Substance); substance-induced psychotic disorders (e.g., (291.5 Alcohol, With Delusions; 291.3 Alcohol, With Hallucinations; 292.11 Amphetamine [or Amphetamine-Like Substance], With Delusions; 292.12 Amphetamine [or Amphetamine-Like Substance], With Hallucinations; 292.11 Cannabis, With Delusions; 292.12 Cannabis, With Hallucinations; 292.11 Cocaine, With Delusions; 292.12 Cocaine, With Hallucinations; 292.11 Hallucinogen, With Delusions; 292.12 Hallucinogen, With Hallucinations; 292.11 Inhalant, With Delusions; 292.12 Inhalant, With Hallucinations; 292.11 Opioid, With Delusions; 292.12 Opioid, With Hallucinations; 292.11 Phencyclidine [or Phencyclidine-Like Substance], With Delusions; 292.12 Phencyclidine [or Phencyclidine-Like Substance], With Hallucinations; 292.11 Sedative, Hypnotic, or Anxiolytic, With Delusions; 292.12 Sedative, Hypnotic, or Anxiolytic, With Hallucinations; 292.11 Other [or Unknown] Substance, With Delusions; 292.12 Other [or Unknown] Substance, With Hallucinations)), substance-induced mood disorders (e.g., (291.8 (new code as of Oct. 1, 1996: 291.89) Alcohol; 292.84 Amphetamine [or Amphetamine-Like Substance]; 292.84 Cocaine; 292.84 Hallucinogen; 292.84 Inhalant; 292.84 Opioid; 292.84 Phencyclidine [or Phencyclidine-Like Substance]; 292.84 Sedative, Hypnotic, or Anxiolytic; 292.84 Other [or Unknown] Substance)); alcohol-induced anxiety, depressive, sleep or sexual disorders (e.g., DSM-IV 291.8), alcohol-induced psychoses (DSM-IV 291.3, 291.5), substance-induced sexual dysfunction (e.g., (291.8 (new code as of Oct. 1, 1996: 291.89) Alcohol; 292.89 Amphetamine [or Amphetamine-Like Substance]; 292.89 Cocaine; 292.89 Opioid; 292.89 Sedative, Hypnotic, or Anxiolytic; 292.89 Other [or Unknown] Substance), substance-induced sleep disorders (e.g., 291.82 Alcohol (291.89 before Oct. 1, 2005; 291.8 before Oct. 1, 1996); 292.85 Amphetamine, Caffeine, Cocaine, Opioid, Sedative, Hypnotic, or Anxiolytic, Other [or Unknown] Substance (292.85 before Oct. 1, 2005)), and substance-induced psychiatric disorders not otherwise specified (e.g., 291.9 Alcohol; 292.9 Other substances). This invention is not limited to these specific examples and is extended to analogous disorders under other diagnostic systems, as well as to other drugs or substances (e.g., nicotine, tobacco, methamphetamine) known or found to produce similar psychiatric effects to those examples outlined above.

An above recited method is also useful therapeutically for relapse inhibition (prevention). Here, the utility also involves the treatment of subjective craving symptoms in addition to behavioral measures of relapse.

Multiple administrations are contemplated for such treatments. Those administrations can occur in a single day, over several days, several months and several years to alleviate the symptoms of the condition mediated by binding.

Illustrative mammals treated in accordance with this method include companion animals such as dogs, cats and ferrets, laboratory animals such as rabbits, guinea pigs, mice and rats, and farm animals such as cows, horses, goats and sheep. Of course, primates such as monkeys, apes and humans are also appropriate subjects.

### Rat Studies of Defensive Burying and Alcohol Dependence

### 1. Results and Discussion

N,N-bis(2-methoxyethyl)-3-(4-methoxy-2-methylphenyl)-2,5-dimethyl-pyrazolo[1,5-a]pyrimidin-7-amine (MPZP, below) was synthesized as an illustrative contemplated compound, and was characterized in vitro and in vivo.

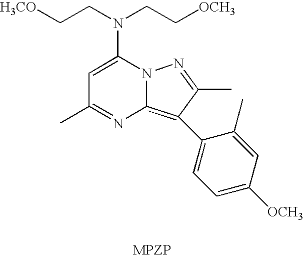

The defensive burying model of active anxiety-like behavior is highly dependent on brain CRF systems [Basso et al., Psychopharmacology (1999) 145:21-30; Diamant et al., Peptides (1992) 13:1149-1158; Korte et al., Physiol. Behay. (1994) 56:115-120; Zorrilla et al., Eur Neuropsychopharmacol. (2003) 13(Suppl. 4):s130-131] and was used to assay the anxiolytic-like properties of MPZP. MPZP then was assayed on a well-established model of alcohol dependence in which rats allowed to self-administer alcohol exhibit enhanced intake following chronic exposure to alcohol vapor (“dependent”) compared to rats not chronically exposed to alcohol vapor (“nondependent”) [Roberts et al., Alcohol Clin. Exp. Res. (1996) 20:1289-1298; Overstreet et al., Alcohol Clin. Exp. Res. (2002) 26:1259-1268; Rimondini et al., FASEB J. (2002) 16:27-35; Valdez et al., Alcohol Clin. Exp. Res. (2002) 26:1494-1501].

This report describes the initial pharmacological and behavioral characterization of a non-peptide small molecule, high affinity CRF₁ specific antagonist, N,N-bis(2-methoxyethyl)-3-(4-methoxy-2-methylphenyl)-2,5-dimethyl-pyrazolo[1,5-a]pyrimidin-7-amine (MPZP). MPZP exhibits a lipophilicity value that is more characteristic of existing CNS-acting drugs and is substantially lower than that of many CRF₁ antagonist predecessors [Zorrilla et al., Exp. Opin. Invest. Drugs (2004) 13:799-828].

The data disclosed hereinafter demonstrate that MPZP has high specificity and affinity for CRF₁, has potent anxiolytic-like activity, and significantly reduces the increased levels of alcohol drinking seen during acute withdrawal in dependent host animals without altering operant responding in nondependent subjects. The results indicate indications for MPZP in further understanding and treating stress-related disorders such as anxiety and alcohol dependence.

The defensive burying model is a test of active anxiety-like behavior [Treit et al., Pharmacol. Biochem. Behay. (1981) 15:619-626; De Boer et al., Eur. J. Pharmacol. (2003) 463:145-161] and has been validated by several anxiolytic and anxiogenic compounds [Korte et al., Physiol. Behay. (1994) 56:115-120; De Boer et al., Eur. J. Pharmacol. (2003) 463:145-161]. Defensive burying is highly dependent on the extrahypothalamic CRF system [Basso et al., Psychopharmacology (1999) 145:21-30; Korte et al., Physiol. Behay. (1994) 56:115-120].

CRF administration increases defensive burying in rats [Diamant et al., Peptides (1992) 13:1149-1158], and CRF antagonists block this response [Basso et al., Psychopharmacology (1999) 145:21-30]. Thus, the ability of MPZP to robustly attenuate burying behavior in the present study confirms a specific role of CRF₁ in mediating defensive burying behavior [Zorrilla et al., Eur Neuropsychopharmacol. (2003) 13(Suppl. 4):s130-1319 and suggests that MPZP is a potent anxiolytic-like drug.

MPZP also significantly reduced excessive drinking during withdrawal in alcohol-dependent animals similarly to other non-peptide CRF₁ antagonists [Chu et al., Pharmacol. Biochem. Behay. (2007) 86:813-821; Funk et al., Biol. Psychiatry (2007) 61:78-86; Gehlert et al., J. Neurosci. (2007) 27:2718-2726; Sabino et al., Psychopharmacology (2006) 189:175-186] without decreasing alcohol self-administration in nondependent animals. In addition, MPZP had no effect on nondependent binge drinking of sweetened alcohol in another study [Ji et al., (2007) In review], further confirming specificity of this compound for the dependence model.

Withdrawal-induced drinking in dependent animals in the present study is hypothesized to be motivated in part by an attempt to reduce the anxiety-like state associated with withdrawal [Valdez et al., Alcohol Clin. Exp. Res. (2002) 26:1494-1501]. Motivational signs of withdrawal (e.g., anxiety, dysphoria, malaise) are considered important in the maintenance and relapse of alcohol consumption in human alcoholics [Koob, Alcohol Clin. Exp. Res. (2003) 27:232-243; Cappell et al., Drug Alcohol Depend. (1979) 4:15-31; Lowman et al., Addiction (1996) 91(Suppl.):s51-71], arguably more important than physical symptoms of withdrawal. The effects of MPZP on alcohol self-administration may be due, at least in part, to its anxiolytic-like properties.

MPZP presumably affects anxiety-like behavior and alcohol drinking via action on CRF₁ cells of the extrahypothalamic CRF system in the extended amygdala. The CRF peptidergic system is distributed throughout the brain, with high concentrations of cell bodies in the paraventricular nucleus of the hypothalamus and in extrahypothalamic areas of the extended amygdala. Extrahypothalamic CRF cell groups include the bed nucleus of the stria terminalis (BNST) and central (CeA) and basolateral subdivisions of the amygdala [Bloom et al., Regul. Pept. (1982) 4:43-48], regions that are known to mediate anxiety-like behavior [Walker et al., J. Neurosci. (1997) 17:9375-9383].

Acute withdrawal from alcohol is accompanied by increased release of CRF in the CeA [Merlo-Pich et al., J. Neurosci. (1995) 15:5439-5447; Zorrilla et al., Psychopharmacology (2001) 158:374-381] and lateral BNST [Olive et al., Alcohol Clin. Exp. Res. Pharmacol. Biochem. Behay. (2002) 72:213-220] as well as increased anxiety-like behavior [Baldwin et al., Psychopharmacology (1991) 103:227-232; Rassnick et al., Brain Res. (1993) 605:25-32]. Administration of nonspecific CRF receptor antagonists directly into the CeA reduces anxiety-like behavior [Rassnick et al., Brain Res. (1993) 605:25-32] and decreases excessive alcohol intake [Funk et al., J. Neurosci. (2006) 26:11324-113329 associated with acute withdrawal in dependent rats.

In summary, the invention contemplates a compound class with high affinity and specificity for CRF₁ receptors. Systemic pretreatment with MPZP, an illustrative member of the compound class, reduced anxiety-like behavior in the defensive burying model and also reduced alcohol self-administration in alcohol-dependent rats.

This compound is also believed to have more general applications. CRF and its receptors are hypothesized to play a critical role in addiction to other drugs of abuse. Withdrawal from chronic nicotine, opiates, cannabinoids, and cocaine elicits increased release of CRF in the CeA and/or increased anxiety-like behavior [Contarino et al., Proc. Natl. Acad. Sci. USA (2005) 102:18649-18654; George et al., Proc. Natl. Acad. Sci. USA (2007), in press; Heinrichs et al., Behay. Pharmacol. (1995) 6:74-80.; Rodriguez et al., Science (1997) 276:2050-2054; Zorrilla et al., Psychopharmacology (2001) 158:374-381; Weiss et al., Ann. N. Y. Acad. Sci. (2001) 937:1-26]. Many drug withdrawal-induced changes can be reversed by CRF antagonists [Weiss et al., Ann. N. Y. Acad. Sci. (2001) 937:1-26]. Altogether, the findings indicate that MPZP and related compounds have therapeutic use in pathological anxiety and drug addiction.

### 1.1. Synthesis and In Vitro Characterization of MPZP

The formulas below compare the structure of MPZP with those of the pyrazolopyrimidine DMP904 and another widely studied CRF₁ antagonist, the pyrrolopyrimidine CP-154,526. Like the other ligands, MPZP has a heterocycle “core” unit and

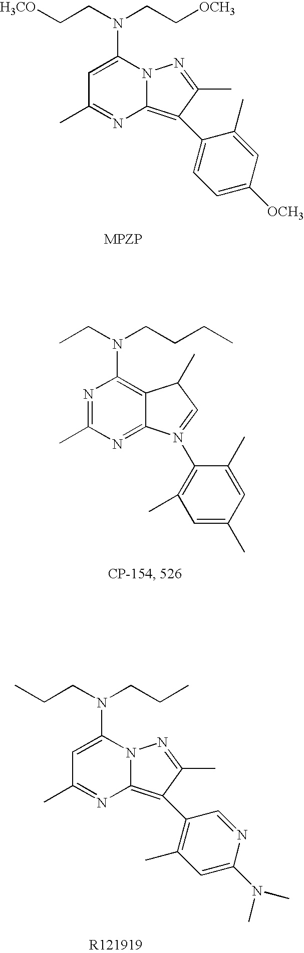

a confirmation-stabilizing ortho- and para-substituted “down” phenyl unit. Unlike DMP904 and CP-154,526, however, MPZP includes polar methoxy substituents in the “top” branched alkyl chains, intended to yield a compound with more “drug-like” lipophilicity [Zorrilla et al., Exp. Opin. Invest. Drugs (2004) 13:799-828].

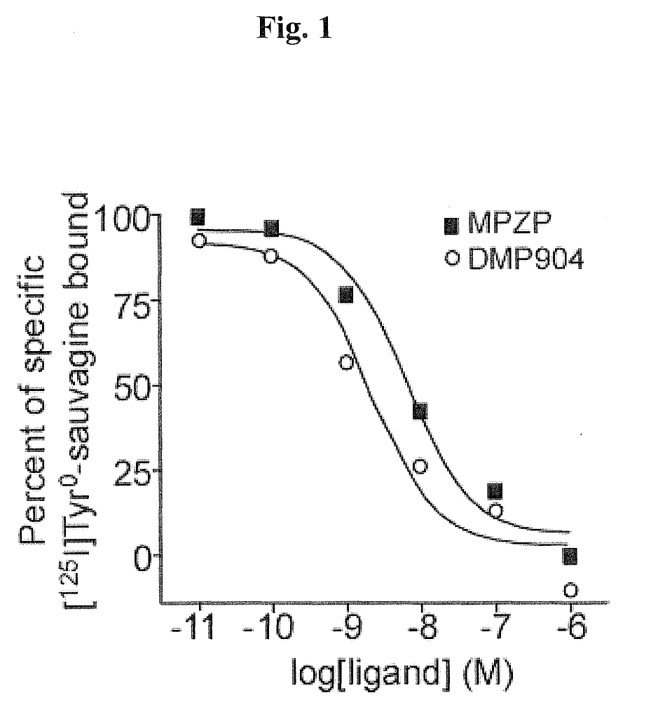

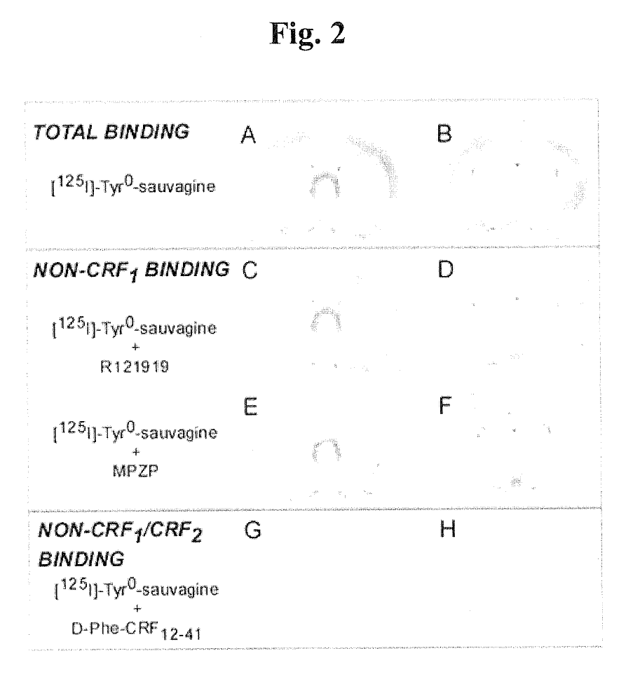

FIG. 1 shows that MPZP displaced specific [¹²⁵I]-Tyr⁰-sauvagine binding from rat cerebellar homogenates on a similar order of potency as DMP904 (PIC₅₀=8.21+0.18 vs. 8.67+0.27, or IC₅₀=6.1 vs. 2.1 nM, respectively), indicating that MPZP is a high-affinity CRF receptor ligand. Specificity of MPZP for CRF₁ vs. CRF₂ receptors was determined via receptor autoradiography (FIG. 2) in which 3 μM MPZP did not displace[¹²⁵I]-Tyr⁰-sauvagine binding from rat lateral septum or ventromedial hypothalamus, choroid plexus, or cerebral arterioles, regions that are rich with CRF₂, but not CRF₁, receptors [Grigoriadis et al., Mol. Pharmacol. (1996) 50:679-686; Heinrichs et al., Neuropsychopharmacology (2002) 27:194-202].

In contrast, MPZP displaced most [¹²⁵I]-Tyr⁰-sauvagine binding from cortex and basolateral amygdala, regions which contain abundant levels of CRF₁ receptors. Thus, MPZP has high specificity for CRF₁ and no measurable specificity for CRF₂ receptors at up to 1 μM concentrations. The pattern of residual [¹²⁵I]-Tyr⁰-sauvagine binding in the presence of MPZP resembled that observed in the presence of R121919, a recognized high-affinity, highly selective CRF₁ antagonist [Heinrichs et al., Neuropsychopharmacology (2002) 27:194-202; Chen et al., J. Med. Chem. (2004) 47:4787-4798] (FIG. 2). Furthermore, when tested at 10 μM concentrations, MPZP did not exhibit high activity at 62 other receptors, transporters, and ion channels in a Novascreen side-effect potential screening assay (GEN SEP1).

Although the binding affinity of MPZP for CRF₁ receptors is slightly less potent than that of those receptors for DMP904 and CP-154,526, MPZP has lipophilicity 2 to 3.5 orders lower than those of these reference compounds and in a range more typical of CNS-acting therapeutics (compare cLogP and cLogD across compounds, Table 1, below) [Zorrilla et al., Exp. Opin. Invest. Drugs (2004) 13:799-828]. The

TABLE 1
Selected physiochemical properties of
MPZP and reference CRF₁ antagonists*

| | MPZP | DMP904 | CP-154,526 |
|---|---|---|---|
| CAS registry | 202579-76-8 | 303579-74-6 | 157286-86-7 |
| number | | | |
| cLogP | 2.95 ± 1.13 | 4.80 ± 1.10 | 6.63 ± 1.30 |
| cLogD, pH 7 | 2.93 | 4.80 | 6.15 |
| pKₐ | 5.32 ± 0.30 | 4.46 ± 0.40 | 7.20 ± 0.30 |
| Polar surface | 61.1 | 51.5 | 29.0 |
| area (Å²) | | | |
| Molar volume | 346.2 ± 7.0 | 311.2 ± 7.0 | 342.0 ± 7.0 |
| (cm³/mol) | | | |

*Physiochemical properties were calculated using Advanced Chemistry Development (ACD/Labs) Software v.8.14 for Solaris (ACD/Labs). CAS, Chemical Abstracts Service.

molecular volume and polar surface area of MPZP, like the other CRF₁ ligands, are consistent with an absorbable, blood-brain barrier-penetrating molecule [Kelder et al., Pharm. Res. (1999) 16:1514-1519.; Zhao et al., J. Chem. Inf. Model (2007) 47:170-175; Fu et al., Pharmazie (2005) 60:354-358; Liu et al., Drug Metab. Dispos. (2004) 32:132-139].

### 1.2. Study 1-Effect of MPZP on Anxiety-Like Behavior

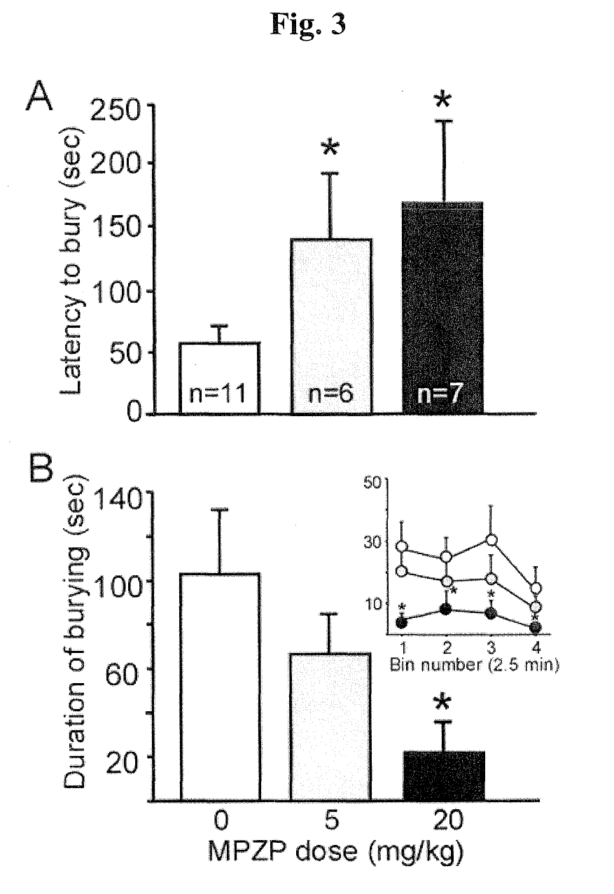

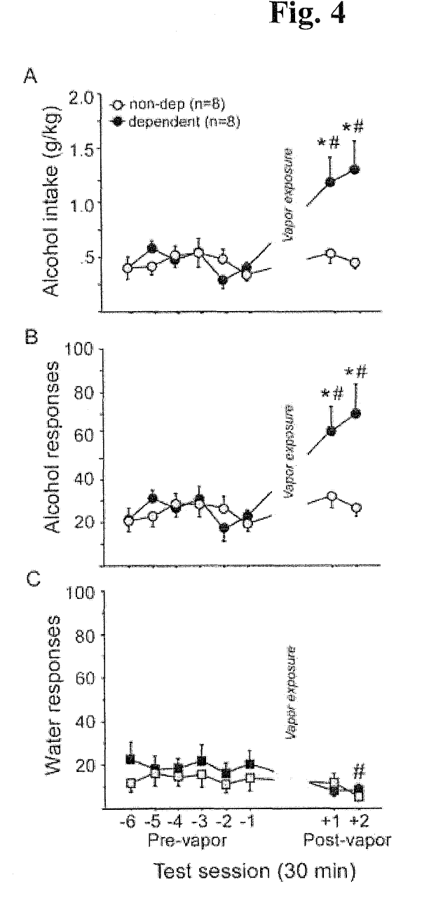

A model of active anxiety-like behavior highly regulated by the CRF system Treit et al., Pharmacol. Biochem. Behay. (1981) 15:619-626; De Boer et al., Eur. J. Pharmacol. (2003) 463:145-161; Korte et al., Physiol. Behay. (1994) 56:115-120] was used to assess the anxiolytic properties of MPZP (FIG. 4). MPZP significantly reduced the latency to bury F_{(2,23)}=4.64, P=0.04, with post hoc analyses showing that both the 5 and 20 mg/kg doses of MPZP increased the latency to start burying compared to vehicle (0 mg/kg) pretreatment (FIG. 3A).

Systemic pretreatment with MPZP also dose-dependently reduced the total duration of defensive burying behavior (F_{(2,23})=3.63, P=0.04). As shown in FIG. 3B, post hoc analyses indicated that the 20 mg/kg dose of MPZP significantly reduced the duration of burying across the 10-minute observation period compared to vehicle (0 mg/kg) pretreatment. Thus, MPZP, a CRF₁ ligand, potently decreased shock-elicited active anxiety-like behavior in the defensive burying test, supporting proposed anxiolytic properties of this compound.

### 3.3. Study 2-Effect of MPZP on Excessive Drinking in an Animal Model of Alcohol Dependence

FIG. 4 illustrates alcohol and water self-administration behavior before and after dependence induction via chronic intermittent alcohol vapor exposure. Post-vapor testing was conducted when dependent animals were in acute withdrawal (6-8 hours after removal from vapors). The increased responding for alcohol observed at this time-point in dependent animals is consistent with previous studies of the dependence model during acute (2 hour) [Roberts et al., Alcohol Clin. Exp. Res. (1996) 20:1289-1298; O'Dell et al., Alcohol Clin. Exp. Res. (2004) 28:1676-1682; Funk et al., J. Neurosci. (2006) 26:11324-11332], 6-8 hour [Sabino et al., Psychopharmacology (2006) 189:175-186], or protracted 2-week [Roberts et al., Neuropsychopharmacology (2000) 22:581-594] withdrawal from alcohol vapors.

There were main effects of Vapor treatment (g/kg intake: F_{(1,98 )}=4.790, P=0.04, FIG. 4A) and Test number (g/kg intake: F_{(7,98)}=7.04, P<0.0001, FIG. 4A; F_{(7,98)}=7.51, P<0.0001, FIG. 4B) on alcohol self-administration, and an interaction of the two factors (g/kg intake: F_{(7,98)}=5.76, P<0.0001, FIG. 4A; alcohol responses: F_{(7,98)}=4.87, P<0.0001, FIG. 4B]. Post hoc analyses indicated that post-vapor g/kg intake and lever responses for alcohol were higher in dependent animals compared to both nondependent animals and pre-vapor responding (FIG. 4A and 4B, all Ps<0.05). Pre-vapor alcohol self-administration was not different between the two groups (Ps>0.05, FIG. 4A and 4B).

Although self-administration of water was slightly, but significantly, lower post-vapor (main effect of test number, F_{(2,28)}=6.36, P=0.005; all pre-vapor tests>post-vapor test 2, P<0.05, FIG. 4C), water responses did not differ between the two groups before or after vapor exposure (P>0.05, FIG. 4C). The data demonstrate that chronic intermittent alcohol vapor exposure in dependent animals elicits increased alcohol drinking during acute withdrawal.

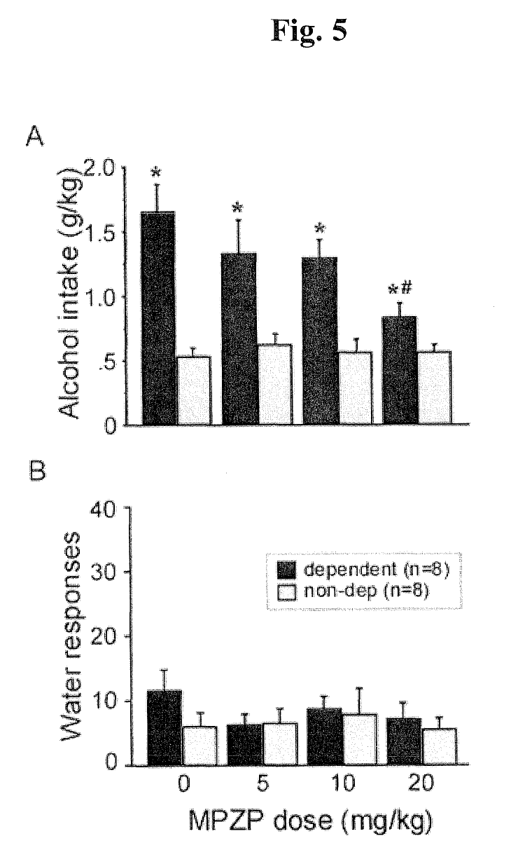

FIG. 5 illustrates the effect of MPZP on alcohol (g/kg intake) and water (responses) self-administration in dependent and nondependent animals. Overall, dependent animals self-administered significantly more alcohol than nondependent animals (main effect of Vapor treatment: F_{(1,42)}=32.61, P<0.0001, FIG. 5A]. In addition, there was a main effect of MPZP (F_{(3,42)}=3.07, P=0.03) and a Vapor treatment x Dose interaction (F_{(3,42)}=3.30, P=0.03; 0 mg/kg dose vs. 20 mg/kg dose, P=0.005, dependent group only) on alcohol self-administration (g/kg intake, FIG. 5A).

Linear contrast analyses detected a Vapor treatment x Dose interaction (F_{(1,14)}=6.31, P=0.02), such that MPZP dose-dependently reduced alcohol self-administration (g/kg intake) in dependent animals (F(1,7)=6.87, P=0.03) but not nondependent animals (F(1,7)=0.01, P=0.95, FIG. 5A). Sigmoidal regression showed a significant sigmoidal dose-response fit to the MPZP-induced reduction of alcohol self-administration in dependent animals (r²=0.907, P<0.05; ED₅₀=10.68 mg/kg MPZP). MPZP had no effect on water self-administration in either dependent or nondependent animals.

### Withdrawal Studies in Nicotine Dependent Rats

The general hypothesis tested here is that chronic nicotine use recruits a major brain stress system, the extrahypothalamic corticotropin releasing factor (CRF) system [Koob et al., Nature Neuroscience (2005) 8:1442-1444, Heinrichs et al., J. Pharmacol. Exp. Ther. (2004) 311:427-440; Zorrilla et al., Expert. Opin. Investig. Drugs. (2004) 13:799-828; Sarnyai et al., (2001) Pharmacol Rev (2001) 53:209-244; Bruijnzeel et al., Neuropsychopharmacology (2006) 32:955-963], which contributes critically to the motivation to continue tobacco use. To this end, the following were examined: whether 1) nicotine withdrawal activates the CRF system in the central nucleus of the amygdala, 2) CRF overactivity, via CRF type 1 receptors (CRF₁), induces an anxiety-like state, a component of the negative emotional state hypothesized to drive nicotine dependence, and 3) nicotine abstinence increases the motivation to take nicotine, via a CRF₁-dependent mechanism.

### Results

Precipitated withdrawal increases CRF levels in the central nucleus of the amygdala. To test the hypothesis that nicotine withdrawal activates the extrahypothalamic CRF system, CRF levels in the central nucleus of the amygdala were measured using in vivo microdialysis and radioimmunoassay. CRF levels were assessed before and after precipitated withdrawal, by administrating mecamylamine to block nicotine receptors in rats with chronic administration of nicotine (nicotine-dependent rats) or saline (non-dependent rats), delivered by osmotic minipumps [Epping-Jordan et al., Nature. (1998) 393:76-79]. In dependent rats, mecamylamine (below) robustly increased CRF-like

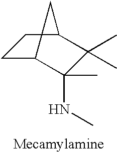

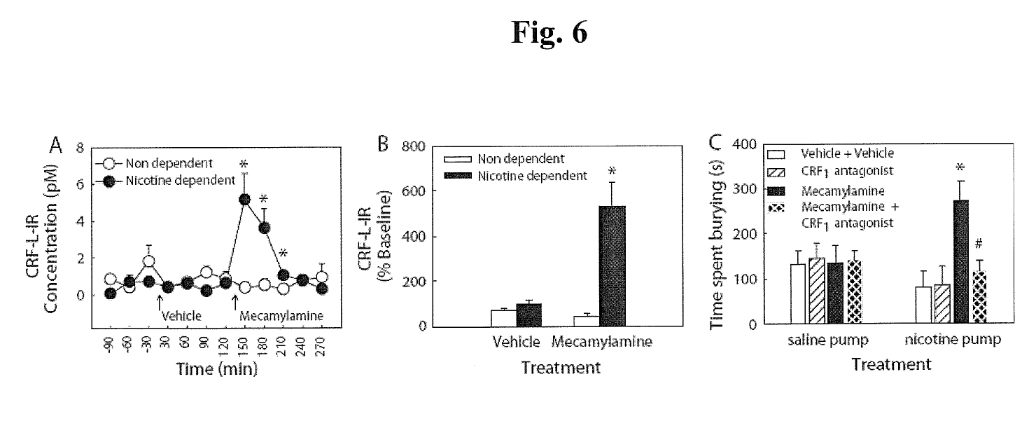

immunoreactivity (CRF-L-IR) in the central amygdala (by over 500% compared with baseline, FIG. 6B), with levels returning to baseline after 2 hours (FIG. 6A). This increase was not observed in saline-treated rats, injected with mecamylamine, and baseline levels did not differ between the two groups.

Precipitated withdrawal increases anxiety-like behavior via activation of CRF₁ receptors. To test the hypothesis that withdrawal-induced increases in CRF activity, via activation of the CRF₁ receptor might be a mechanism responsible for the appearance of a negative emotional state, anxiety-like behavior during precipitated withdrawal was measured in nicotine-dependent rats and non-dependent rats, using the defensive burying test [Basso et al., Psychopharmacology (Berl). (1999) 145:21-30; De Boer et al., Eur. J. Pharmacol. (2003) 463:145-161].

In dependent rats, mecamylamine injection increased the time spent burying (+243%), and decreased the latency to bury (−70%) compared to vehicle injection, two markers of active anxiety-like behavior [De Boer et al., Eur. J. Pharmacol. (2003) 463:145-161] (FIG. 6C), without affecting general activity (rearing), non-anxiety behaviors (resting, grooming) or a passive form of anxiety-like behavior (freezing). Mecamylamine injection did not alter anxiety-like behavior in non-dependent rats, consistent with the differential effects of mecamylamine on extracellular amygdala levels of CRF-L-IR (FIGS. 6A, 6B). Pre-treatment with a selective small molecule non-peptide CRF₁ receptor antagonist (N,N-bis(2-methoxyethyl)-3-(4-methoxy-2-methylphenyl)-2,5-dimethyl-pyrazolo [1,5-a]pyrimidin-7-amine (MPZP), 4 mg/kg s.c.) [De Boer et al., Eur. J. Pharmacol. (2003) 463:145-161; Richardson et al., Program No. 783. 4 Neuroscience Meeting Planner. Atlanta, Ga.: Society for Neuroscience, OnlineI (2006)], blocked the anxiogenic-like effect of mecamylamine to increase burying in nicotine-dependent rats (FIG. 6C).

To confirm that increases of CRF in the central nucleus of the amygdala elicit anxiety-like behavior, anxiety-like behavior was measured using the defensive burying test, after bilateral infusion of CRF (30 pmol total dose) or vehicle in the central nucleus of the amygdala of naive rats one minute before beginning of the burying test. CRF administered directly into the amygdala increased the time spent burying and decreased the latency to bury during the first 5 minutes after infusion (Table 2),

TABLE 2

| | | Latency to | | |
|---|---|---|---|---|
| | Burying (s) | Bury (s) | Grooming (s) | Rearing (s) |
| Vehicle | 0.1 ± 0.2 | 341 ± 78 | 21 ± 17 | 103 ± 15 |
| CRF (30 | 7.1 ± 4.7 * | 165 ± 91 * | 30 ± 27 | 91 ± 15 |
| pmol) | | | | |

* p < 0.05 vs. vehicle. (s) = seconds.

without affecting other behaviors (rearing, grooming, resting and freezing). The low burying baseline observed in these animals can be explained by the extensive handling, and the higher body weight of the rats (˜600 g) in this study, two factors known to decrease baseline level of burying [Diamant et al. Peptides (1992) 13:1149-1158; Pare et al., Physiol Psychol. (1969) 69:214-218]. Such a low baseline allows for anxiogenic-like effects to be detected more easily, and has been reported previously in young rats under different conditions [Richardson Endocrinology (2006 May) 147(5):2506-2517. Epub 2006 Feb. 2.].

The data in Table 3, below, compare the effects of systemic administration of the CRF1 antagonist MPZP on behavior in the defensive burying test in rats implanted with nicotine or saline pumps, some of which were precipitated into withdrawal by a nicotine receptor antagonist. MPZP is shown to blunt the anxiogenic-like effects of mecamylamine precipitated withdrawal from nicotine.

TABLE 3

| | | | CRF1 | | CRF1 |
|---|---|---|---|---|---|
| | | Vehicle + | antagonist + | Vehicle + | antagonist + |
| | | Vehicle | Vehicle | Mecamylamine | Mecamylamine |
| | Treatment | Data are in seconds | | | |
| Burying | nicotine | 79 ± 36 | 85 ± 42 | 271 ± 46* | 115 ± 24 |
| | saline | 133 ± 29 | 145 ± 34 | 135 ± 39 | 143 ± 20 |
| Latency to | nicotine | 104 ± 31 | 130 ± 36 | 31 ± 9* | 82 ± 19^{#} |
| bury | saline | 84 ± 17 | 104 ± 29 | 87 ± 25 | 80 ± 18 |
| Freezing | nicotine | 1 ± 1 | 13 ± 7 | 1 ± 1 | 7 ± 7 |
| | saline | 5 ± 4 | 9 ± 8 | 1 ± 1 | 5 ± 3 |
| Grooming | nicotine | 17 ± 9 | 48 ± 18 | 16 ± 5 | 38 ± 14 |
| | saline | 16 ± 9 | 26 ± 12 | 30 ± 12 | 15 ± 5 |
| Rearing | nicotine | 205 ± 22 | 181 ± 21 | 105 ± 25 | 126 ± 21 |
| | saline | 179 ± 21 | 183 ± 19 | 118 ± 14 | 139 ± 7 |
| Resting | nicotine | 4 ± 3 | 24 ± 11 | 49 ± 21 | 68 ± 26 |
| | saline | 40 ± 16 | 11 ± 7 | 67 ± 24 | 51 ± 22 |

*p < 0.05 vs. vehicle + vehicle
^{#}p, 0.05 vs. vehicle + mecamylamine

Thus, increased CRF release, and CRF₁ receptor activation during abstinence appears to mediate anxiety-like behavior during precipitated withdrawal in nicotine-dependent rats. This hypothesis predicts that, in dependent rats, a period of abstinence may lead to an increase in nicotine intake during the subsequent access to nicotine, and that blocking the action of CRF using a CRF₁ receptor antagonist could prevent this increase in nicotine intake.

Abstinence increases nicotine intake in rats given extended access to self-administration. To evaluate the effect of abstinence on nicotine intake an animal model of intermittent exposure to 23 hours extended access to nicotine self-administration was used. The intermittent access consisted of 4 consecutive days of self-administration at a constant unit dose (0.03 mg/kg/injection) followed by 3 days of abstinence because 3 days of abstinence from chronic nicotine administration increases anxiety-like behavior in rats [Irvine et al., Pharmacol. Biochem. Behay. (2001) 68:319-325; Irvine et al., Behay. Pharmacol. (1999) 10:691-697].

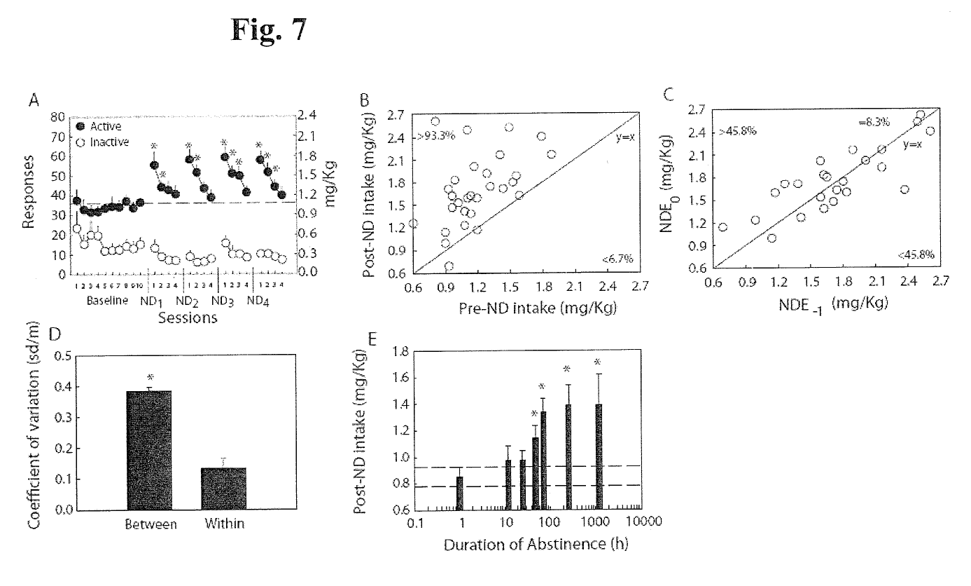

Nicotine intake significantly increased during the first session following each cycle of abstinence (FIG. 7A), and returned to baseline by the 4^{th} day of nicotine self-administration. The “nicotine deprivation effect” reflected mainly increased drug intake during the active (dark) period, which represents about 80% of daily nicotine intake, but a significant increase, albeit smaller, was also observed during the light period (data not shown). Rats exhibited “drug-loading” behavior during the beginning of the active period such that the non-deprived baseline amount of intake normally requiring 12 hours was attained in only 6.4±1.2 hours.

Scatter plot of pre-versus post-abstinence nicotine intake shows that the majority (>93%) of post-abstinence nicotine intakes were higher than pre-abstinence nicotine intakes, demonstrating the robustness of the phenomenon (FIG. 7B). The fact that post-abstinence nicotine intakes measured during the four successive cycles were i) highly correlated with each other (mean r=0.81, range: 0.72 to 0.92, all p<0.05, FIG. 7C), ii) evenly distributed around the y=x line, and iii) that the coefficient of variation between subjects was three times higher than the coefficient of variation within subjects (FIG. 7D) demonstrate existence of reliable inter-individual differences in the effect of abstinence on nicotine intake.

The time course of appearance of the nicotine deprivation effect was evaluated by exposing rats to different durations of abstinence, from 1 hour to about 2 months (1201 hours). Abstinence-induced increase in nicotine intake was significant after 48 hours, reached a maximum after 3 days of abstinence, and remained elevated even after 2 months of abstinence (FIG. 7E).

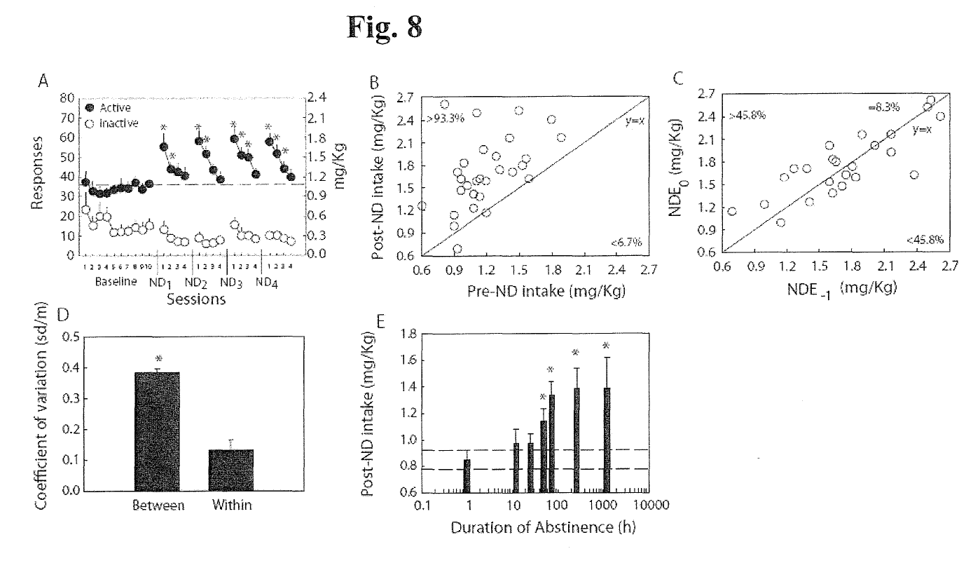

To test the relevance of the nicotine deprivation effect to nicotine dependence, the effect of 3 days of abstinence was tested in rats given limited access to nicotine self-administration (1 h/session), a condition known not to induce any spontaneous signs of withdrawal [Paterson et al., Psychopharmacology (Berl). (2004) 173:64-72]. Abstinence was found to have no effects in rats with limited access (FIG. 8B), whereas as observed in the previous experiments, abstinence markedly increased nicotine responding in rats with extended access (23 hour/session) (FIG. 8A). Inactive lever responses were not affected by abstinence.

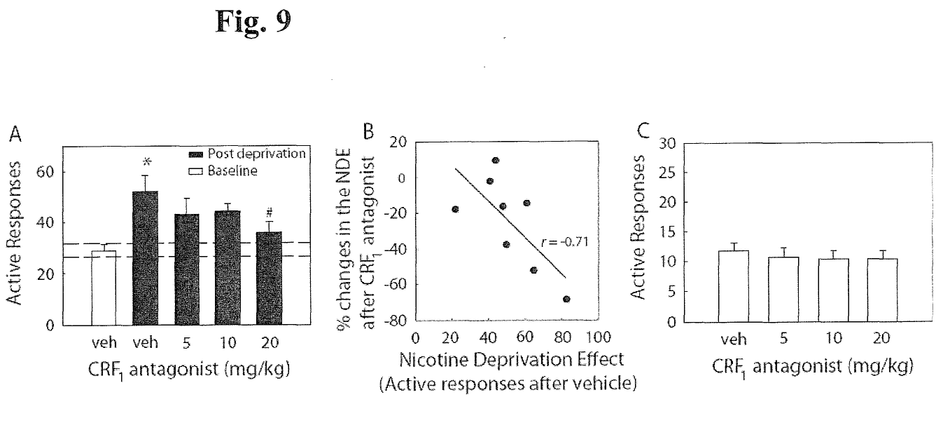

Antagonism of CRF₁ receptor prevents abstinence-induced increases in nicotine intake. To evaluate the role of the CRF-CRF₁ system in the nicotine deprivation effect, the effect of the CRF₁ receptor antagonist, MPZP, on nicotine responding was tested in rats with intermittent access to extended nicotine self-administration (23 hour, 4 days/week). Following abstinence, pre-treatment with the CRF₁ antagonist dose-dependently decreased nicotine intake (FIG. 9A) compared with vehicle-treated rats, and blocked the nicotine deprivation effect compared with baseline levels. As expected, the CRF₁ receptor antagonist decreased nicotine self-administration during the active (dark) period when abstinence-induced escalation of nicotine intake occurred, but not during the inactive (light) period (data not shown). Efficacy of the CRF₁ receptor antagonist correlated with the magnitude of the nicotine deprivation effect observed in any given subject (FIG. 9B). CRF₁ receptor antagonist efficacy did not correlate with the magnitude of baseline responding (r=0.05, not significant) and had no effect in rats given limited access to nicotine (1 hour) (FIG. 9C), supporting a specific relation to abstinence responding and nicotine dependence.

### Discussion

The results here show that precipitated withdrawal, in nicotine dependent rats, increases CRF release in the central nucleus of the amygdala, and increases anxiety-like behavior via a CRF₁-dependent mechanism. Nicotine abstinence produces a robust increase in nicotine intake, in rats allowed extended access to nicotine self-administration. Finally, the increased nicotine intake can be blocked by pre-treatment with a specific CRF₁ receptor antagonist.

Nicotine withdrawal, precipitated by mecamylamine, increased CRF release in the central nucleus of the amygdala, in rats chronically exposed to nicotine. Interstitial amygdalar CRF concentration reached a maximum 30 minutes after mecamylamine injection, with levels returning to baseline after 2 hours. This pattern may be explained by the short pharmacokinetic half-life of mecamylamine (about 1 hour) [Debruyne et al., J. Pharm. Sci. (2003) 92:1051-1057], and the constant exposure to nicotine. Withdrawal-induced CRF release in nicotine dependent rats, and intra-amygdala infusion of CRF in naive rats, were both associated with an increase in time spent burying, and a decreased latency to bury in the defensive burying test, whereas the CRF₁ receptor antagonist MPZP reversed the increase in defensive burying observed during mecamylamine-precipitated nicotine withdrawal.

The defensive burying test possesses face and predictive validity as an animal model of normal and pathological anxiety; in particular, time spent burying reflects an active coping strategy to an anxiogenic environment, and is respectively decreased and increased by anxiolytic- and anxiogenic-like compounds [Basso et al., Psychopharmacology (Berl). (1999) 145:21-30; De Boer et al., Eur. J. Pharmacol. (2003) 463:145-161; Gilligan et al., Bioorganic & Medicinal Chemistry (2000)8:181-189]. Administration of mecamylamine, MPZP, or CRF did not change general activity or non-anxiety behaviors such as rearing, resting and grooming. This argues against non-specific effects, and confirming an earlier report showing that CRF infusion in the central nucleus of the amygdala does not alter feeding or grooming behavior [Jochman et al., Behay. Neurosci (2005) 119:1448-1458]. Also, the increase of defensive burying observed after mecamylamine or CRF administration was not associated with an increase in the time spent freezing. Freezing under these conditions may represent a different measure of anxiety related to passive and not active-avoidance, and can be dissociated pharmacologically from the active form of anxiety measured by the time spent burying or the latency to bury [De Boer et al., Eur. J. Pharmacol. (2003) 463:145-161].

It is thus likely that the CRF-CRF1 system is not involved in all aspects of negative emotions, and also that some forms of anxiety-like behavior may be unchanged during nicotine withdrawal. The CRF system has been implicated in anxiety-like behavior, and studies of CRF₁ receptor antagonists have promising potential for anxiolytic drug development [Zorrilla et al., Expert. Opin. Investig. Drugs. (2004) 13:799-828]. The findings with nicotine discussed here add to reports showing increased amygdalar CRF release, and anxiety-like behaviors following withdrawal from other drugs of abuse including ethanol, cocaine, opiates and cannabinoids [Weiss et al., Ann. N. Y. Acad. Sci. (2001) 937:1-26; Funk et al., J Neurosci (2006) 26:11324-11332; Merlo et al., J Neurosci (1995) 15:5439-5447; Contarino et al., Proc. Natl. Acad. Sci. U.S.A. (2005) 102:18649-18654; Bruijnzeel et al., Brain Research Reviews (2005) 49:505-528; and Ambrosio et al., Synapse (1997) 25:272-276], and suggest that over-activation of the extrahypothalamic CRF-CRF₁ system may constitute a common denominator of motivational aspects of drug withdrawal. Overactivation of the CRF-CRF₁ system during withdrawal is also associated with a hypoactivation of the dopaminergic system in the central nucleus of the amygdale [Panagis et al., Synapse (200) 35:15-25], suggesting that both systems may interact to mediate anxiety-like behavior during withdrawal. However, whether the increase in CRF and the decrease in dopamine are causally linked is unknown and needs further investigation.

An escalating dose regimen of nicotine associated with intermittent abstinence periods was recently shown to produce high levels of nicotine intake, suggesting that abstinence may increase subsequent nicotine intake [O'dell et al., Pharmacol. Biochem. Behay. (2007) 86:346-353]. New data presented herein extend this finding by showing that at a constant unit dose, three days of forced abstinence induces a marked increase of nicotine intake. The nicotine deprivation effect was mainly observed during the early active period (dark). This situation is very similar to the human condition where abstinence is followed by an increase in smoking, during the early active period (light), followed by a titration period of nicotine intake [Benowitz et al., Clin. Pharmacol. Ther. (1984) 35:499-504; Isaac et al., Nature. (1972) 236:308-310]. The time course of recovery to the original basal levels of intake if deprivation is not initiated has not been fully investigated, but preliminary results suggest that recovery time will depend on the duration of withdrawal, the magnitude of the deprivation effect, the number of self-administration sessions, and the period of nicotine intake (light vs. dark).

The nicotine deprivation effect was found to be a long-lasting phenomenon that progressively develops during the first week of abstinence, and remains robust for at least 2 months. The time course of the nicotine deprivation effect is similar to the phenomenon of incubation of reward craving [Lu et al., Neuropharmacology (2004) 47 Suppl 1:214-226] where an increase in responding for cues related to drug delivery after withdrawal has been observed across several drugs (cocaine, heroin, methamphetamine), and natural rewards (sucrose) as well [Lu et al., Neuropharmacology (2004) 47 Suppl 1:214-226; Grimm et al., Nature (2001) 412:141-142; Shalev et al., Psychopharmacology (Berl) (2001) 156:98-107; Shepard et al., Biol. Psychiatry (2004) 55:1082-1089; and Tran-Nguyen et al., Neuropsychopharmacology (1998) 19:48-59]. However, this increase in responding is observed under extinction and reinstatement sessions, but not after re-exposure to the drug itself [Lu et al., Neuropharmacology (2004) 47 Suppl 1:214-226], and there is little evidence to date suggesting that the incubation effect leads to increased drug self-administration.

The present results demonstrate that the potential for nicotine self-administration progressively develops during withdrawal, and leads to increased nicotine intake during renewed drug access. In this regard, the nicotine deprivation effect maybe more comparable to the alcohol deprivation effect [Heyser et al, Alcohol Clin. Exp. Res. (1997) 21:784-791].

Moreover, reliable inter-individual differences were observed in the magnitude of the nicotine deprivation effect, suggesting that this measure may represent a relevant marker of individual vulnerability to nicotine dependence. This hypothesis is supported by the fact that the nicotine deprivation effect was not observed in rats with limited (1 hour) access to nicotine; a condition known not to induce spontaneous signs of withdrawal, a central aspect of nicotine dependence [Paterson et al., Psychopharmacology (Berl). (2004) 173:64-72], but a condition sufficient to produce the reward incubation effect.

The nicotine deprivation effect is unlikely to result from a sensitized reward state for nicotine or a loss of tolerance to the effect of nicotine during withdrawal (both of which would have led to a decrease in nicotine intake compared with baseline) but may be better explained by a negative reinforcement construct. Here, dependent rats may be hypothesized to escalate their nicotine intake after abstinence to obtain relief from a resulting CRF-CRF₁-mediated anxiety-like state.

The role of the CRF-CRF₁ system in the nicotine deprivation effect was confirmed by the experiment showing that the increased nicotine intake observed after abstinence was dose-dependently blocked by pre-treatment with MPZP. This result is reinforced by the fact that CRF₁ receptor antagonist was more effective at reducing nicotine intake in individual animals exhibiting a high nicotine deprivation effect, again suggesting that the magnitude of the nicotine deprivation effect may be a marker of individual vulnerability to nicotine dependence.

The potential role of CRF₂ receptors in these effects is currently unknown and would require further investigation. However, inactivation of CRF₂ receptor is more likely to produce a stress-like response than an anti-anxiety-like effect based on pharmacological and knockout studies [Bale et al., Nat Genet. (2000) 24:410-414; Valdez et al., Brain Res. (2003) 980:206-212]. Antagonism of CRF₁ receptors prevents deficits in brain reward function [Bruijnzeel et al., Neuropsychopharmacology (2006) 32:955-963] and increased in anxiety-like behavior associated with precipitated nicotine withdrawal. Thus, MPZP administration may be hypothesized to block abstinence-induced increases in nicotine intake through a reduction of negative reward and anxiety-like states contributing to the negative emotional state associated with nicotine abstinence.

Taken together these results suggest that a key mechanism in nicotine dependence is withdrawal-induced overactivation of the CRF-CRF₁ receptor system, which contributes to the increased negative emotional state that drives subsequent nicotine intake. The recruitment of such a negative emotional system may explain one site of vulnerability for the transition from nicotine use to nicotine dependence, and suggests a new target for non-nicotine pharmacotherapy for tobacco addiction.

### Attenuation of Escalated Cocaine

### Self-administration in Rats

Previous studies have shown that CRF₁ antagonists have differential effects on ethanol self-administration in dependent vs. nondependent rats [Funk et al., Biol. Psychiatry (2007) 61:78-86; Sabino et al., Psychopharmacology (2006) 189:175-186; Heilig et al., Trends Neurosci. (2007) 30:399-406]. Therefore, the present study tested the hypothesis that CRF₁ receptor blockade differentially affects rats with a history of short and extended access to cocaine. To test the hypothesis, the effect of two non-peptide CRF₁ receptor antagonists, antalarmin and N,N-bis(2-methoxyethyl)-3-(4-methoxy-2-methylphenyl)-2,5-dimethyl-pyrazolo[1,5a]pyrimidin-7-amine (MPZP), was examined on cocaine self-administration under short access (ShA, 1 hour/day) and long access (LgA, 6 hours/day) conditions. The CRF₁ receptor antagonists more effectively decrease the increased cocaine intake of LgA rats than the intake of ShA rats.

Results

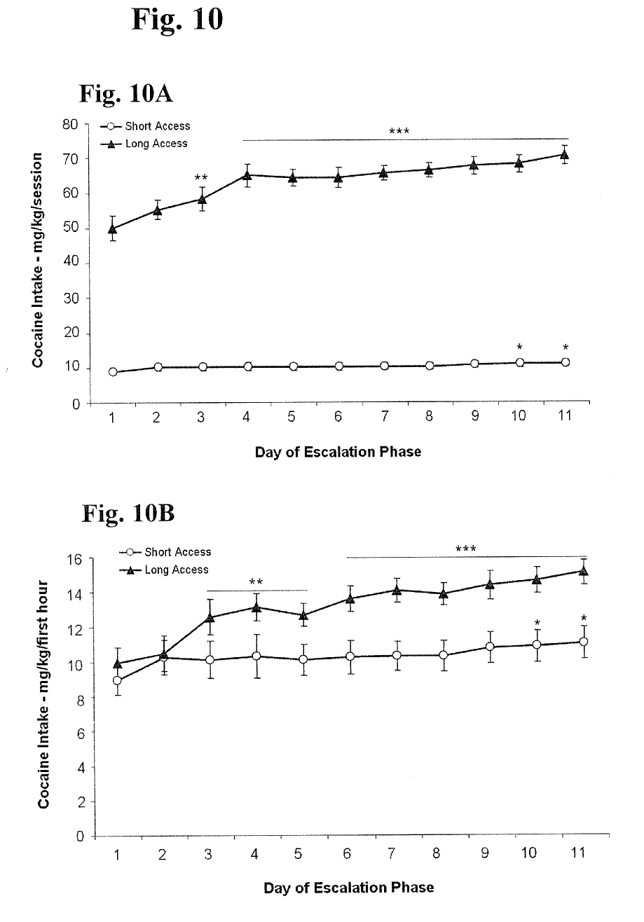

FIGS. 10A and 10B illustrate cocaine intake (mg/kg) for ShA and LgA groups during the entire session and first hour, respectively. Data analyses of cocaine intake for the ShA group and the first hour of the LgA group during the first 11 days of escalation revealed a main effect of daily sessions (F_{10,300}=9.031 p<0.0001), main effect of access (F_{1,30}=7.347, p<0.05), and an overall interaction (F_{10,300}=2.996, p<0.01). Compared to escalation session 1, the ShA group exhibited increased responding in sessions 10-11, whereas the LgA group exhibited increased 1 hour responding earlier, beginning in session 3.

LgA animals also significantly increased total session responding by session 3. Cocaine intake within the first hour by LgA rats significantly exceeded that of ShA rats on Day 5 (p<0.01), continuing through sessions 6 to 11 (p<0.001). Separation of the cocaine intake data into the respective antagonist treatment groups yielded the following results: The Antalarmin groups (ShA vs. LgA) exhibited a main effect for daily sessions (F_{10,150}=6.091, p<0.0001), an overall interaction (F_{10,150}=2.632, p<0.01), but no main effect for group on cocaine intake. The antalarmin LgA group increased intake by Day 2 for the first hour and by Day 4 for total session intake. The MPZP groups (ShA vs. LgA) exhibited a main effect of daily sessions (F_{13,130}=4.465, p<0.0001), a main effect for group (F_{1,13}=5.758, p<0.05), and an overall interaction (F_{13,130}=2.343, p<0.05) on cocaine intake. The MPZP LgA group increased intake by Day 10 for the first hour and by Day 3 for the total session intake. The separation of cocaine intake data into the respective antagonist treatment groups resulted in a lack increased intake for either ShA group.

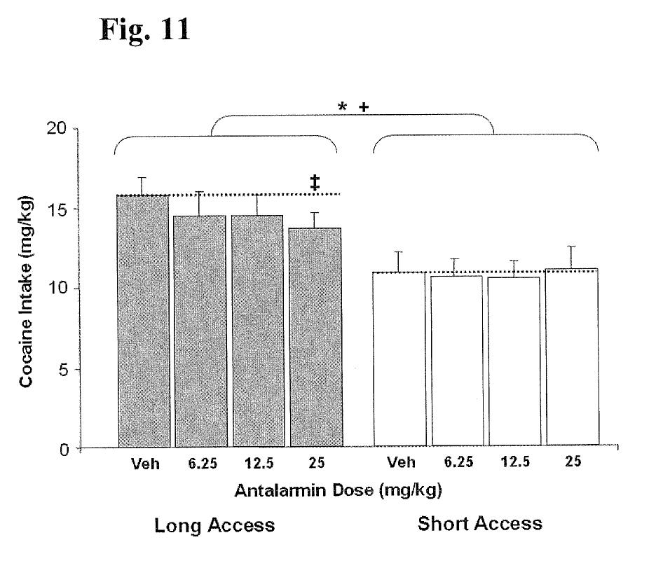

The two CRF₁ antagonists differentially decreased cocaine intake (mg/kg) during the test sessions. Two-way ANOVA of cocaine intake following antalarmin pretreatment (FIG. 11) revealed a main effect of Access (F_{1,15}=5.446, p<0.05), but no main effect for antalarmin dose and no interaction. However, a linear contrast two-way ANOVA showed a significant Dose×Access interaction (F_{1,15}=5.05, p<0.05), indicating that the strength of the log-linear relation of antalarmin dose to cocaine intake differed significantly between access conditions. To interpret the linear contrast Dose×Access interaction, a follow-up linear dose analyses on each access group, followed by pair-wise, within-subjects Dunnett's tests vs. vehicle, showed that the LgA group exhibited a significant log-linear dose-dependent trend toward deceased intake (F_{1,24}=7.1592, p<0.05, slope=−0.32), with pair-wise comparisons indicating a significant decrease (13.3%) at the 25 mg/kg dose compared to vehicle pretreatment.

In contrast, no dose-dependent trend was evident in the ShA group (F_{1,2}=0.02, p>0.05, slope=0.01394), and pair-wise comparisons showed that no dose significantly altered cocaine intake compared to vehicle pretreatment (FIG. 11). Additionally, there was a simple main effect of dose of antalarmin on cocaine intake in LgA rats (F_{3,45}=3.11, p<0.05) with a significant decrease of cocaine intake at 25 mg/kg compared with the vehicle. In contrast, no simple main effect of dose on cocaine intake was found in ShA rats.

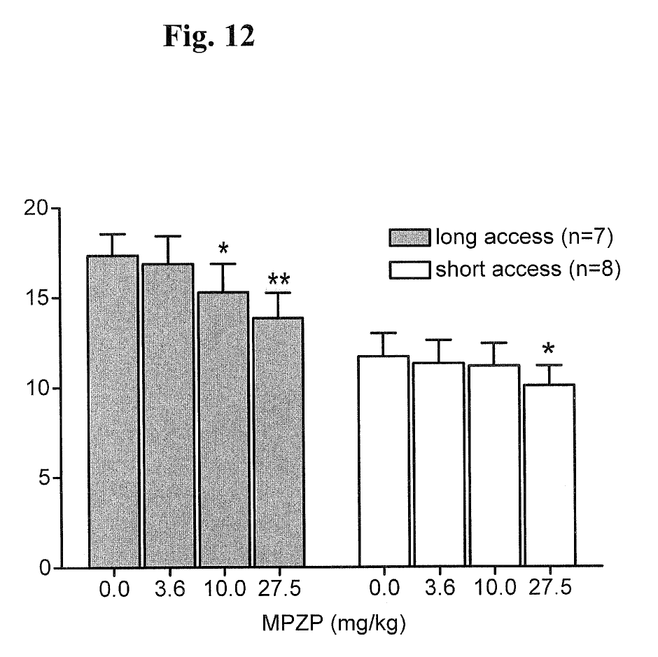

In contrast to antalarmin, MPZP (FIG. 12) significantly altered cocaine intake in both ShA and LgA rats, albeit to different degrees. Two-way ANOVA revealed an overall main effect for access (F_{1,13}=7.249, p<0.05), a main effect for MPZP dose (F_{3,39}=10.076, p<0.001), but no interaction. However, a two-way linear contrast showed a significant dose×access linear contrast interaction (F_{1,13}=5.48, p<0.05). Linear trend dose effects of MPZP were present in the LgA (F_{1,18}=17.07, p<0.001) and SgA groups (F_{1,21}=10.57, p<0.01), when considered separately. Additionally, there was a simple main effect of dose on cocaine intake both in ShA and LgA rats (LgA, F_{3, 39}=9.31, p<0.01; ShA, F_{3,39}=4.08, p<0.05). Post hoc Dunnett's test showed a significant decrease of cocaine intake at 10 mg/kg (p<0.05) and 27.5 mg/kg (p<0.01) in LgA rats and at 27.5 mg/kg in ShA rats (p<0.05).

The slopes of the linear trend dose functions were significantly less than zero for both LgA (F_{1,26}=11.60, p=0.002) and ShA rats (F_{1,30}=6.02, p=0.02), but tended to be steeper in the LgA than ShA group (F_{1,56}=3.01, p=0.08, slopes=−0.601±0.176 vs. −0.254±0.106, respectively). This steeper slope for the LgA group suggests a greater effectiveness, or capacity, of MPZP to reduce cocaine self-administration in LgA rats than ShA rats (FIG. 12).

Discussion

Anxiety and dysphoria occur during cocaine abstinence in human cocaine users [Kampman et al., Addict. Behay. (1998) 23:449-461]. This negative emotional state is hypothesized to motivate the maintenance and persistence of drug intake via negative reinforcement mechanisms, thereby playing a critical role in the development of drug dependence [Koob et al., Science (1997) 278:52-58]. Evidence supports the notion that the CRF system mediates anxiety and other dysphoric states [for review, see Zorrilla et al., Expert Opin. Investig. Drugs (2004) 13:799-828; Bale et al., Annu. Rev. Pharmaco. Toxicol. (2004) 44:525-557], and recruitment of the CRF system has been hypothesized to be involved in drug dependence in humans [Koob, Ann. N. Y. Acad. Sci. (1999a) 897:27-45]. The present study tested whether rats with a history of extended access to cocaine and resultant escalation of intake showed increased sensitivity to the ability of CRF₁ antagonists to reduce cocaine self-administration, as would be predicted with increased activity of the CRF receptor system.

Consistent with previous reports, LgA rats increased cocaine intake during the first hour and across the total 6 hours across test sessions [Ahmed et al., et al. Science (1998) 282:298-300; Wee et al. (2007) J. Pharmacol. Exp. Ther. (2007) 320:1134-1143]. In humans, drug intake increases with the development of drug dependence [American Psychiatric Association (2000) Diagnostic and statistical manual of mental disorders, 4th edition. American Psychiatric Press, Washington D.C.], supporting the face validity of the extended access cocaine self-administration procedure as a model of cocaine dependence in humans.

Under the present test conditions, when all ShA rats were combined, increased cocaine intake was observed across the escalation period, but to a much smaller degree and more slowly than the increase seen in first hour intake in LgA rats. The hypothesis of differential activity of CRF systems in rats with a history of cocaine escalation was supported by the current findings.

Systemic pretreatment with the non-peptide CRF₁ antagonist antalarmin dose-dependently decreased cocaine intake selectively in LgA rats, leading to a significant decrease in self-administration at the 25 mg/kg dose. A previous study found that 30 minute pretreatment of antalarmin did not alter the dose-response function of cocaine self-administration in rhesus monkeys with limited (2 hour), daily drug access [Mello et al., Pharmacol. Biochem. Behay. (2006) 85:744-751], which is consistent with the present finding that antalarmin had no effect in ShA rats. Similar to the present results, antalarmin (20 mg/kg, i.p.) dose-dependently reduced escalated ethanol intake in ethanol-dependent rats tested during acute withdrawal, yet did not influence ethanol self-administration in nondependent rats [Funk et al., Biol. Psychiatry (2007) 61:78-86].

Doses of antalarmin, similar to those used in the present study (20 mg/kg, i.p.), were shown previously to inhibit spontaneous defensive withdrawal behavior as well as intracerebroventricular CRF-induced anxiogenic-like behavior in the elevated plus maze [Zorrilla et al., Brain Res. (2002) 952:188-199]. These data support the hypothesis that the CRF system contributes to the escalated drug intake of rats with extended drug access, a model of the development of drug dependence.

The non-peptide CRF₁ antagonist MPZP decreased cocaine intake in both ShA and LgA rats. However, MPZP reduced cocaine self-administration in LgA rats at a lower dose than in ShA rats, suggesting that cocaine intake by LgA rats was more sensitive to the blockade of CRF₁ receptors than the intake by ShA rats. Doses of MPZP similar to those used in the present study also reduced anxiety-like behavior in the defensive burying test [Fekete et al., Society for Neuroscience (2003) Program No. 538.13. Abstract Viewer and Itinerary Planner, Washington, D.C.]. An increased time spent burying defensively in the shock-probe test was previously found in LgA rats compared with ShA rats after 22 days of cocaine self-administration with extended access, which lasted over a month (Aujla et al., 2005). Thus, the data suggest that the physiological systems affected by cocaine self-administration with extended access may reflect a hypersensitive state in the CRF system, which produces an increased anxiety-like state upon the exposure to shock stimulus in the defensive burying test and perhaps contributes to an increased cocaine intake.

The decreased cocaine intake in ShA rats resulting from MPZP pretreatment at the highest dose was somewhat unexpected because previous reports using various doses and regimens of antalarmin, etomide, ketoconazole, astressin, and dexamethasone in rhesus monkeys, and CP 154,526 in Wistar rats, did not find a relationship between the CRF system and the acute reinforcing effects of cocaine assessed by self-administration and discriminative stimuli [Mello et al., Pharmacol. Biochem. Behay. (2006) 85:744-751; Broadbear et al., J. Pharmacol. Exp. Ther. (1999) 290:1347-1355; Przegalinski et al., Neuropeptides (2005) 39:525-533]. However, consistent with the present results, CP-154,526, a non-peptide CRF₁ receptor antagonist, reduced cocaine self-administration (0.5 mg/kg/injection) and cocaine-induced conditioned place preference in rats [Lu et al., J. Neurochem. (2003) 84:1378-1386; Goeders et al., Neuropsychopharmacology (2000) 23:577-586].

Possible explanations for the difference of CRF antagonist effects include the doses of antagonists used and the session durations. There was some, albeit modest, escalation in the ShA rats in the present study. Goeders and Guerin [Neuropsychopharmacology (2000) 23:577-586] used a higher dose of CP-154,526 (25 mg/kg) than that used (20 mg/kg) by Przegalinski et al., Neuropeptides (2005) 39:525-533. The physiological systems affected by escalated cocaine intake in LgA groups may provide for a hypersensitive state such that a lower dose of CRF₁ antagonist would have more of an effect on the system.

The pharmacological differences between antalarmin and MPZP on cocaine intake by ShA rats might be explained by pharmacokinetic variables related to the compounds' different lipophilicities, or the different routes and vehicles of administration. Antalarmin, a pyrrolopyrimidine, is approximately 4.5 orders (30,000-fold) more hydrophobic than MPZP (cLogP=6.98 vs. 2.52, respectively; Advanced Chemistry Development Software Solaris V4.67). The very high lipophilicity of antalarmin results in poor aqueous solubility and low bioavailability of the compounds, with a pharmacokinetic profile unfavorable for accumulation of high central levels [Zorrilla et al., Expert Opin. Investig. Drugs (2004) 13:799-828]. The use of hydroxypropyl βas a vehicle excipient also may have increased central availability of MPZP by increasing solubility and distribution and, perhaps, reducing degradation [Strickley, (2004) Pharm. Res. (2004) 21:201-230].

Neuroadaptation in the CRF system in the extended amygdala has been proposed to drive the negative motivational state associated with abstinence in drug-dependent humans [Koob, Eur. Neuropsychopharmacol (2003) 13:442-452]. Research substantiating this hypothesis includes findings that extracellular CRF levels are increased in the central amygdala during cocaine, ethanol, cannabinoid, and opioid withdrawal in rats [Richter et al., Synapse (1999) 32:254-261; Merlo-Pich et al. J. Neurosci. (1995) 15:5439-5447; Rodriguez de Fonseca et al., Science (1997) 276:2050-2054; Weiss et al., Ann. N. Y. Acad. Sci. (2001) 937:1-26, respectively], and that tissue content levels of CRF in the amygdala are depleted during withdrawal from cocaine or ethanol [Zorrilla et al., Psychopharmacology (2001) 158: 374-381; Funk et al. J. Neurosci. (2006) 26:11324-11332]. CRF antagonists have been found to reduce negative emotional states during withdrawal from cocaine [Basso et al., Psychopharmacology (1999) 145:21-30; Przegalinski et al., Neuropeptides (2005) 39:525-533], methamphetamine [Moffett et al., Psychopharmacology (2007) 190:171-180], nicotine [Bruijnzeel et al., Neuropsychopharmacology (2007) 32:955-963], and ethanol [Baldwin et al., Psychopharmacology (1991) 103:227-232; Rassnick et al., Brain Res. (1993) 605:25-32; Menzaghi et al., Ann. N. Y. Acad. Sci. (1994) 739:176-184]. Such negative emotional states may indicate decreased brain reward function, which has been observed in previous studies in LgA rats. Ahmed et al., Nat. Neurosci. (2002) 5:625-626 showed progressively increasing current thresholds for intracranial self-stimulation during withdrawal from daily, extended drug access, findings indicative of decreased brain reward function. Altogether, these results support an overall hypothesis that CRF system activity is increased in the extended amygdala which, in turn, may contribute to escalation of cocaine intake in LgA rats.

Rather than an effect on extrahypothalamic CRF systems, a possible alternative explanation for the current findings is that antalarmin and MPZP reduced cocaine intake because of an ability of CRF₁ antagonists to reduce HPA activation. For example, the structurally related compound DMP904 inhibited ACTH release from rat pituitary corticotropes (Li et al., 2005) and dose-dependently inhibited the stress-induced increase of plasma corticosterone in rats [Lelas et al., J. Pharmacol. Exp. Ther. (2004) 309:293-302]. Similarly, antalarmin (20 mg/kg, i.p.) blocked CRF-induced increases in plasma ACTH levels (Webster et al., 1996). The understanding of the relationship between the acute reinforcing effect of cocaine and the HPA axis is evolving [Marinelli et al., (1997) J. Pharmacol. Exp. Ther. (1997) 281:1392-1400]. Mantsch et al., J. Pharmacol. Exp. Ther. (1998) 287:72-80 reported that dexamethasone, a glucocorticoid receptor agonist, inhibited the acquisition of cocaine self-administration in rats, yet corticosterone treatment promoted acquisition. Later studies found no clear relationship between glucocorticoids and stress-induced escalation of cocaine intake, cocaine-seeking behavior, or the discriminative stimulus properties of cocaine in rats [Mantsch et al., Neuropsychopharmacology (2006) 32:367-376; Mantsch et al., Pharmacol. Biochem. Behay. (1999) 64:65-73]. Similarly, Broadbear and colleagues [J. Pharmacol. Exp. Ther. (1999) 290:1347-1355] reported that cocaine-maintained responding was not altered by effective inhibitors of HPA axis hormonal responses in rhesus monkeys. Furthermore, under conditions similar to those in the present study, Mantsch et al., Psychoneuroendocrinology (2003) 28:836-862 noted the development of tolerance in HPA axis responses to cocaine intake in LgA rats, but not in ShA rats. Thus, cocaine exposure may initially activate the HPA axis [Marinelli et al., Eur. J. Neurosci. (2002) 16:387-394], whereas prolonged cocaine exposure may ultimately result in a subsequent blunting of the HPA axis and sensitization of extrahypothalamic CRF systems [Lee et al., Brain Res. (1994) 666(1):93-98; Pecina et al., BMC Biol. (2006) 4:8; Shepard et al., Behay. Brain Res. (2006) 174:193-196].

The present study demonstrates that CRF₁ receptor antagonists decreased cocaine intake in rats, especially in those with a history of extended, as opposed to brief, daily cocaine access. The data suggest that neuroadaptations in the CRF system partly underlie the increased motivation to self-administer cocaine that develops during psychostimulant dependence.

### Rat Studies of Defensive Burying and Alcohol Dependence

### 2. Materials and Methods

### 2.1. Animals

Adult male Wistar rats were obtained from Charles River Laboratory (Kingston, N.Y.). Rats were housed 2-3 per cage with food and water available ad libitum. Lights were on a 12 h light/dark cycle, with lights on at 0600. For the behavioral studies, animals were allowed 4-7 days of acclimation to the laboratory and were frequently handled prior to the start of both experiments. Brain tissue for receptor binding and autoradiography assays was obtained from alcohol-naive rats that were anesthetized with isofluorane and immediately decapitated. Brains were rapidly removed and snap-frozen in isopentane (2-methylbutane, Sigma, St. Louis, Mo.) and stored at −80° C. for autoradiography or placed immediately on an ice-cold stage with whole cerebellum obtained for receptor binding assays. All procedures met the guidelines of the National Institutes of Health Guide for the Care and Use of Laboratory Animals and were approved by the Institutional Animal Care and Use Committee at The Scripps Research Institute.

2.2 Synthesis and in vitro characterization of N,N-bis(2-methoxyethyl)-3-(4-methoxy-2-methylphenyl)-2,5-dimethyl-pyrazolo[1,5-a]pyrimidin-7-amine (MPZP)

MPZP was synthesized as described in Gilligan et al., Bioorg. Med. Chem. (2000) 8:181-189 and Arvanitis et al., WO 9803510, published 29 Jan. 1998.

Binding activity of MPZP was determined in a competition assay using [¹²⁵I]Tyr^{C}-sauvagine (2200 Ci/mmol; Perkin Elmer, Waltham, Mass.) as the radioligand. Cerebellum was homogenized in homogenizing buffer (Dulbecco's phosphate-buffered saline [PBS]:1.5 mM KH₂PO₄, 8.1 mM Na₂HPO₄, 2.7 mM KCl, 138 mM NaCl, pH 7.2, supplemented with 10 mM MgCl₂, 2 mM EGTA) using a Polytron (Dispersing and Mixing Technology, Kinematica, Littau-Lucerne, Switzerland) at setting 6 for 2×15 seconds on ice. The homogenate was centrifuged at 45,000×g for 20 minutes at 4° C. The pellet was resuspended and spun at 45,000×g for 20 minutes at 4° C. The final pellet was resuspended in assay buffer (homogenizing buffer supplemented with protease inhibitor; 1 tablet/10 ml; Sigma CAT#58829-20TAB, St. Louis, Mo., pH 7.4) using a Polytron.

The reaction was initiated by adding 0.05 ml of [¹²⁵I]Tyr⁰-sauvagine to 1.5 ml polypropylene tubes containing 0.1 ml of membrane preparation (about 2 mg protein/ml) and 0.05 ml of a CRF₁ antagonist at logarithmic interval concentrations from 10⁻⁶ to 10⁻¹¹ M. MPZP binding affinity was compared to that of DMP904, a structurally related reference compound known to exhibit high, selective affinity for CRF₁ receptors [Gilligan et al., Bioorg. Med. Chem. (2000) 8:181-189], whose structural formula is shown below.

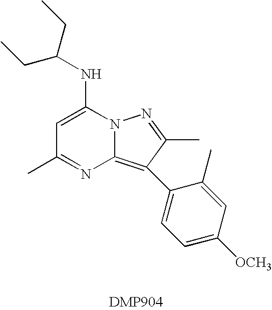

Total binding was determined using assay buffer in lieu of a CRF₁ antagonist, and nonspecific binding was determined in the presence of 1 μM unlabeled sauvagine. The final radioligand concentration was 0.2 nM, and the reaction was incubated at room temperature for 2 hours. The reaction tubes were centrifuged at 12,000 rpm for 5 minutes to terminate the reaction. The supernatant was removed and the pellets washed twice with ice-cold washing buffer (DPBS with 0.01% Triton-X100). Tubes then were centrifuged at 12,000 rpm, and the supernatant was removed. The pellet-containing tip was cut off and counted in an automated 10-detector gamma counter (MicroMedic Apex, ICN Biomedical, Costa Mesa, Calif.) at 80% efficiency. Six independent radioligand displacement assays were performed in each of which the total radioligand bound was less than 10% of the total amount of radioligand added to the tube.

Specificity of MPZP for other receptor, transporter, and ion channel targets was determined at 1 and 10 μM concentrations via a commercial screening service (Novascreen, GEN SEP I panel, Hanover, Md.).

For CRF receptor autoradiography, brain tissue was sectioned coronally (20 μm) using a cryostat (−17° C.). Sections were mounted on Superfrost Plus+ charged glass slides (Fisher Scientific, Pittsburgh, Pa.), permitted to dry completely, and stored in airtight boxes at −80° C. until the day of autoradiography. Autoradiography was performed using standard procedures based on the previous characterization of [¹²⁵I]Tyr⁰-sauvagine [Grigoriadis et al., Mol. Pharmacol. (1996) 50:679-686].

Slides containing triplicate adjacent brain sections were thawed to room temperature and permitted to dry completely. Each section then was outlined using a PAP pen (Calbiochem, San Diego, Calif.). Sections were incubated in assay buffer (DPBS with 10 mM MgCl₂, 2 mM EGTA, 1 tablet/100 ml protease inhibitor, 0.15% bovine serum albumin) for 15 minutes to remove endogenous ligand. Slides then were incubated under one of four conditions: (1) 0.2 nM [¹²⁵O]Tyr⁰-sauvagine to determine total binding; (2) 0.2 nM radiolabeled sauvagine+1 μM R121919 (below)

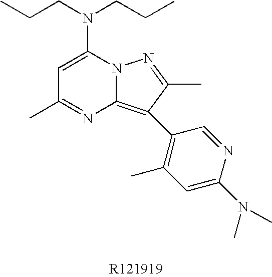

to determine non-CRF₁ (e.g., CRF₂) receptor binding; (3) 0.2 nM radiolabeled sauvagine+3 μM MPZP to determine non-CRF₁ (e.g., CRF₂) receptor binding using the experimental compound under study; (4) 0.2 nM radiolabeled sauvagine+0.3 μM unlabeled D-Phe-CRF₁₂₋₄₁, a subtype-nonspecific CRF receptor antagonist, to determine non-CRF₁/CRF₂ (e.g., nonspecific binding).

After 2 hours incubation at room temperature, unbound radioligand was removed via a brief dip in ice-cold assay buffer, followed by two 5 minute rinses in ice-cold washing buffer (DPBS with 0.01% Triton-X100) and one brief dip in ice-cold distilled, deionized H₂O. Slides then were dried at room temperature and exposed to Kodak Biomax MR film for 2 days. Unlabeled peptides (sauvagine, D-Phe-CRF₁₂₋₄₁) were generously provided by Dr. Jean Rivier (The Salk Institute, La Jolla, Calif.). Images were captured using a light box and digital camera computer workstation using a MTI CCDC72 digital camera equipped with a 90 mm Tamron macro lens. The frame-grabber software was Scion FGC Capture, and image analysis was performed with ImageJ 1.39 (National Institutes of Health, Washington, D.C.).

### 2.3. MPZP Preparation

MPZP [compound 146(bd) in Table 1 of WO 98003510 at page 113] was prepared for systemic administration by first solubilizing it in 1 M HCl (10% final volume). It then was diluted using 25% w/v hydroxypropyl β-cyclodextrin (HBC, Cargill, Cedar Rapids, Iowa) (80% final volume) and back titrated under constant mixing, with descending concentrations of NaOH (2, 1, 0.1 M) (10% final volume) resulting in a final suspension of 10 mg/ml MPZP in 20% HBC (pH 4.5). Lower concentrations then were prepared by serial dilution with vehicle (20% HBC, pH 4.5). Animals were administered the appropriate dose via a 2 ml/kg injection (0-20 mg MPZP/2 ml 20% HBC vehicle/kg body weight).

### 2.4. Study 1-Effect of MPZP on Anxiety-like Behavior

The defensive burying test was used to assess effects of MPZP on anxiety-like behavior [Treit et al., Pharmacol. Biochem. Behay. (1981) 15:619-626; De Boer et al., Eur. J. Pharmacol. (2003) 463:145-161]. This model has been validated by anxiolytic and anxiogenic compounds, which decrease and increase defensive burying behavior, respectively [Korte et al., Physiol. Behay. (1994) 56:115-120; De Boer et al., Eur. J. Pharmacol. (2003) 463:145-161]. For two consecutive days before defensive burying testing, animals were acclimated to the testing apparatus by placing them for 45 minutes in the testing cage (a polycarbonate rat housing cage with 2 cm of bedding covering the floor and a small hole centered on a long dimension of the cage 1 inch above the bedding to accommodate the shock probe on the subsequent test day). On the day of testing, animals were brought into the anteroom at least 2 hours before testing began.

Subjects were subcutaneously pretreated with MPZP (0, 5, 20 mg/kg) in a between-subjects design 1 hour before their test session. For testing, animals were placed individually in the test cage, and a shock probe connected to a Coulbourn precision shocker (model E13-01, Coulbourn Instruments, Allentown, Pa.) delivered one 1.5 mA shock (lasting <1 s) upon contact. As soon as the animal was shocked (verified by a startle response), the shock current was deactivated, and the 10 minute test began.

Contact with the shock probe under these conditions results in the rat displacing bedding material with treading-like movements of the forepaws and shoveling movements of the head, often directed toward the shock probe. Latency to the first display of burying behavior and time spent burying (in four 2.5 minute bins throughout the 10 minute test) were assessed [Korte et al., Physiol. Behay. (1994) 56:115-120]. Defensive burying testing occurred 2-6 hours into the dark cycle, during the rat's active phase when defensive burying behavior is high. This time point was selected to allow for measurable decreases in burying behavior following administration of MPZP.

Tests were recorded, and two reliable raters naive to the treatment conditions of the animals independently scored burying behavior of each subject (r=0.97, total duration; r=0.87, latency to bury). Rater averages were used in statistical analysis. A total of 24 rats (MPZP doses: 0 mg/kg, n=11; 5 mg/kg, n=6; 20 mg/kg, n=7) were used for this experiment.

### 2.5. Study 2-Effect of MPZP on Excessive Drinking in an Animal Model of Alcohol Dependence

The effect of MPZP on drinking behavior was studied in an established animal model of alcohol dependence. In this model, rats previously trained to self-administer alcohol exhibit increased anxiety-like behavior and enhanced alcohol intake during withdrawal from chronic, intermittent alcohol exposure (dependent) compared to rats not chronically exposed to alcohol vapor (nondependent) [O'Dell et al., Alcohol Clin. Exp. Res. (2004) 28:1676-1682; Funk et al., J. Neurosci. (2006) 26:11324-11332; see also Roberts et al., Alcohol Clin. Exp. Res. (1996) 20:1289-1298; Overstreet et al., Alcohol Clin. Exp. Res. (2002) 26:1259-1268; Rimondini et al., FASEB J. (2002) 16:27-35; Valdez et al., Alcohol Clin. Exp. Res. (2002) 26:1494-1501 for related models].

### 2.5.1. Acquisition of Operant Alcohol Self-Administration

Animals were permitted to self-administer alcohol or water orally in a concurrent, two-lever, free-choice contingency. A continuous reinforcement (fixed ratio-1) schedule was used in which each lever press was reinforced. Animals acquired alcohol self-administration using a variation of the previously described saccharin fading free-choice operant conditioning protocol [Samson et al., Alcohol Clin. Exp. Res. (1986) 10:436-442].

The present procedure culminates in pharmacologically relevant levels of alcohol self-administration, as defined by blood alcohol levels (BALs), in nondependent animals with limited access to alcohol over a 6-week period [Roberts et al., Alcohol Clin. Exp. Res. (1999) 23:1151-1157]. The modified procedure in the present study utilized a sweetened solution (“supersac”) containing 3% glucose and 0.125% saccharin (Sigma, St. Louis, Mo.) instead of water restriction and 0.2% saccharin to initiate and maintain operant responding [Funk et al., J. Neurosci. (2006) 26:11324-11332].

Animals respond for the sweetened solution within 1-2 training sessions, making water restriction unnecessary. Operant sessions during training were conducted 5 days per week between 0900 and 1500 (lights on at 0600). Operant sessions were 30 minutes in duration, except during the initial days of training in which sessions lasted up to 2 hours to permit acquisition of responding for the sweetened solution. Alcohol (10% w/v) then was added to the sweetened solution, and once mean responding stabilized (around one week) the glucose was removed from the solution, leaving only 0.125% saccharin and 10% w/v ethanol.

Animals were kept at this stage until mean responding again stabilized (around 1 week), and saccharin concentrations were gradually reduced in about 50% successive steps over 2-10 days, ultimately leaving an unadulterated 10% w/v ethanol solution. Animals then were maintained on 10% w/v ethanol for at least 3 weeks, and stable responders (±25% across three consecutive sessions) were evenly divided into two groups matched for baseline responding and exposed to intermittent ethanol vapors (dependent) or air (nondependent) as described below. A total of 16 rats (dependent, n=8; nondependent, n=8) were used for this experiment.

### 2.5.2. Operant Self-Administration Apparatus

The self-administration system comprised test chambers (Coulbourn Instruments, Allentown, Pa.) contained within wooden sound-attenuated ventilated cubicles. The test chambers were equipped with two retractable levers located 4 cm above the grid floor and 4.5 cm to either side of a small stainless steel receptacle containing two drinking cups. Two infusion pumps (Razel Scientific Instruments, Stamford, Conn.) were connected to the system so that a lever press resulted in the delivery of 0.1 ml of solution. Tap water was delivered to one dish, and the experimental solution (e.g., sweetened solution or alcohol) was delivered to the other dish. Fluid delivery and recording of operant self-administration were controlled by a computer. Lever presses were not recorded during the 0.5 second inter-response time-out interval when solution was being delivered.

### 2.5.3. Solutions for Oral Self-Administration

Alcohol (10% w/v) was prepared with 95% ethyl alcohol and tap water. Glucose (3%) and/or saccharin (0-0.125%; Sigma, St. Louis, Mo.) was added to the water or alcohol solutions to achieve the appropriate concentration.

### 2.5.4. Dependence Induction by Alcohol Vapor Chambers

A recent modification of the alcohol dependence model was made to reflect the natural progression of alcohol dependence in which alcohol exposure occurs in a series of extended exposures followed by periods of withdrawal [O′Dell et al., Alcohol Clin. Exp. Res. (2004) 28:1676-1682]. Chronic exposure to intermittent alcohol vapor exposure elicits even higher alcohol self-administration than continuous vapor (Ibid.), and the intermittent procedure therefore was used to induce dependence in trained animals in the present study. Vapors were delivered on a 14 hours on/10 hours off schedule for 4 weeks before post-vapor testing began. This schedule of exposure has been shown to induce physical dependence (Ibid.).

In the chambers, 95% alcohol flows from a large reservoir to a peristaltic pump (model QG-6, FMI Laboratory, Fluid Metering Inc., Syosett, N.Y.). Ethanol is delivered from the pump to a sidearm flask at a flow rate that can be regulated. The flask is placed on a heater so that the drops of alcohol hitting the bottom of the flask are vaporized. Air flow controlled by a pressure gauge is delivered to the flask and carries the alcohol vapors to the vapor chamber that contains the animal cages. The flow rate was set to deliver vapors that result in BALs between 0.125-0.250g %.

Beginning 4 weeks after the onset of vapor exposure, post-vapor alcohol self-administration testing was conducted 2 times per week during acute withdrawal (6-8 hours after cessation of daily vapor exposure). For testing the effects of MPZP on self-administration behavior, subjects were subcutaneously pretreated with MPZP (0, 5, 10, 20 mg/kg) 1 hour before their 30 minute test session in a Latin square design with 3-4 days between tests.

### 2.5.5. Blood Collection and Measurement of Blood Alcohol Levels

Throughout the time in vapors, blood samples were obtained 1-2 times per week to confirm that vapor-exposed animals had BALs between 0.125 and 0.250g %. Blood samples were collected by the tail-snip method (0.1-0.2 ml) from all animals (both ethanol vapor-exposed dependent and control air-exposed nondependent groups) just after the vapors turned off (0800 hours).

Plasma (5 μl) was used for measuring BALs using an Analox AM 1 analyzer (Analox Instruments, Lunenburg, Mass.). The reaction is based on the oxidation of alcohol by alcohol oxidase in the presence of molecular oxygen (alcohol+O₂→acetaldehyde+H₂O₂). The rate of oxygen consumption is directly proportional to the alcohol concentration. Single-point calibrations were done for each set of samples with reagents provided by Analox Instruments (0.025-0.400g %). When dependent animals had BALs outside the 0.125-0.250g % range, the evaporated ethanol values (ml/h) were adjusted to reestablish the correct range. As expected, BALs were always undetectable in nondependent animals, but tail bleeding was performed to control for any stress experienced during this procedure.

### 2.6. Statistical Analyses

For analysis of competition binding assays, four-parameter logistic equations were fit to the mean % specific (Total-nonspecific binding) [¹²⁵I]Tyr⁰-sauvagine binding observed across concentrations of MPZP or the reference CRF₁ antagonist DMP904 in six independent experiments. The effect of MPZP on defensive burying behavior (latency to first bury and burying time) were analyzed by analysis of variance (ANOVA), in which Dose was a between-subjects factor and Time bin was a repeated measure for duration of burying.

Pre-vs. post-vapor operant responding (number of presses for alcohol or water, g/kg alcohol intake) was analyzed by two-way ANOVAs with Test number a within-subjects factor and Vapor treatment a between-subjects factor. The effect of MPZP on operant responding (number of presses for alcohol or water, g/kg alcohol intake) was analyzed by a two-way ANOVA with Dose being a within-subjects factor and Vapor treatment being a between-subjects factor. Linear trend and sigmoidal regression analyses were used to characterize the dose-response curve of MPZP on operant responding. Unless stated otherwise, significant interactions were followed by Bonferroni/Dunn post hoc tests and P≦0.05 was considered statistically significant.

### Withdrawal Studies in Nicotine Dependent Rats

### Materials and Methods

All animal use procedures were approved by The Scripps Research Institute's Animal Care and Use Committee and were in accordance with the National Institutes of Health guidelines. Effort was made to reduce the number of animals by using both between- and within-subject design studies. Adult male Wistar rats (Charles River) were housed in a temperature-controlled vivarium with a 12 hour:12hour light/dark cycle. Tests were performed at the beginning of the dark cycle (10:00 am).

Drugs

Nicotine hydrogen tartrate salt (Sigma, Natick) was dissolved in saline at pH 7.4 and experimenter-administered via minipump or self-administered via indwelling jugular catheter. Doses are expressed as free base. Mecamylamine (Sigma; Natick, Mass.) was dissolved in saline and administered i.p. (1 ml/kg). The CRF₁ antagonist (N,N-bis(2-methoxyethyl)-3-(4-methoxy-2-methylphenyl)-2,5-dimethyl-pyrazolo-[1,5α]pyrimidin-7-amine, or MPZP) was synthesized at The Scripps Research Institute by Dr. P. Wirshing, dissolved in 20% hydroxypropyl beta-cyclodextrin (Cavitron, Cargill) in isotonic saline at pH 4.5 and administered s.c. (2 ml/kg, 45 to 60 min before testing). Rat/human CRF was supplied by Dr. Jean Rivier, The Salk Institute. CRF was dissolved in 1× PBS (pH 7.4) and prepared fresh few minutes before intracerebral injection. The doses and time of injections were selected based on previous studies [Richardson et al., Program No. 783. 4 Neuroscience Meeting Planner. Atlanta, Ga.: Society for Neuroscience, OnlineI (2006); Specio et al., Program No. 777. 11 Neuroscience Meeting Planner. Washington, D.C.: Society for Neuroscience, Online (2004)].

Microdialysis

The changes in interstitial levels of CRF-like-immunoreactivity (CRF-L-IR) in the central nucleus of the amygdala were examined during mecamylamine-precipitated withdrawal (1.5 mg/kg, ip). Rats were subcutaneously implanted with osmotic minipumps (model 2m12, 14 days, 5 μL/hour; Durect Corporation, Palo Alto, Calif.) delivering either saline (non-dependent, n=5) or nicotine (nicotine-dependent, n=7) (3.16 mg/kg/day, free base, s.c.) and a microdialysis guide cannula (SciPro Inc., Sanborn N.Y., USA) stereotaxically positioned 1 mm above the central nucleus of the amygdala using the following coordinates (AP −3.3 mm; ML ±4.2 mm; V −6.5 mm, from dura with flat skull). After 14 days of pump exposure, a microdialysis probe (1 mm PES membrane, 15 kDA MW cutoff; SciPro Inc., Sanborn N.Y., USA) was lowered into the guide cannula and allowed to equilibrate for 12 hours (1 μl/min flow rate, artificial cerebrospinal fluid). Subsequently dialysate samples (30 minute fractions) were collected for a period of baseline sampling and following saline and mecamylamine challenge injections using a within-subjects design. Sample tubes were kept on wet ice during collection and were then frozen on dry-ice until later analysis by RIA.

CRF Immunoassay

Dialysate CRF-like immunoreactivity was quantified with a sensitive and specific solid-phase radioimmunoassay (RIA) adapted from Zorrilla et al. Psychopharmacology (Berl). (2001) 158:374-381 to increase sensitivity. Immulon-4 96 well plates (Dynatech, Chantilly, Va.) were coated with protein A/G (1 μg/100 μl, 1 M NaHC0₃/well, pH 9.0; Calbiochem, La Jolla, Calif.) overnight. Plates were rinsed with wash buffer (0.15 M K₂HPO₄ supplemented with 0.2 mM ascorbic acid and 0.1% Tween-20, pH 7.5) to dislodge loose Protein A/G. Wells were incubated 48 hours at 4° C. with 50 μanti-CRF serum (rC68, generously provided by W. Vale, The Salk Institute, La Jolla, Calif.) at a titer of 1:300,000 in gelatin assay buffer. After three rinses to dislodge loose antibody, 50 μl of dilute sample (in duplicate) or standard (3 to 1000 pg/ml, in quadruplicate) were incubated overnight (about 18 hours) at 4° C. Following incubation, 50 μl of [¹²⁵I-Tyr⁰]r/hCRF (about 4,000 cpm/50 μl; New England Nuclear, Boston, Mass.) were added to each well and incubated for an additional 24 hours at 4° C. Wells were rinsed, blotted dry and separated, and residual radioactivity was counted by a gamma counter for 5 minutes per well. Sensitivity of the assay is approximately 0.1 fmol/well, and inter- and intra-assay coefficients of variation at the ED₅₀ dose range from 7-11%.

Defensive Burying Behavior

Rats were subcutaneously implanted with osmotic minipumps delivering either saline (n=33) or nicotine (n=31) (3.16 mg/kg/day, free base, s.c.) as described above. After 14 days of pump exposure, testing was performed 5-8 hours into the dark cycle in a standard cage with 2 inches of bedding (wood shavings) along the bottom and a small hole centered in one side one inch above the bedding to accommodate the shock-probe. Rats were habituated (45 minutes) to the test cage for 2 days prior to testing. On the test day, mecamylamine or its vehicle were administered 30 minutes before behavioral testing, and the CRF₁ antagonist or its vehicle were administered 45 minutes before behavioral testing (n=7-9 per group). On contact with the probe and shock delivery (using a Coulbourn precision shocker, 1.5 mA, AC, <1 s), verified by a startle response, the probe was deactivated. The latency and duration of probe-directed burying, rearing, resting, grooming, and freezing were measured from videotape over a 10 minutes period by an experimenter blind to the subject treatment condition using a computer program.

Intracerebral Cannulations and CRF Infusions

Rats were anesthetized with an isoflurane-oxygen mixture, and 26-gauge stainless steel guide cannulas (Plastics One, Roanoke, Va.) aimed 2 mm above the central nucleus of the amygdala stereotaxically were implanted bilaterally: AP −2.6 mm; ML ±4.2 mm; V −5.2 mm, from dura, with flat skull (Paxinos and Watson, 1998). The guide cannulas were secured to the skull with dental cement and anchor screws, and guide cannulas were maintained with stylets. Intracerebral injections were administered with the use of injectors (33-gauge; Plastics One) that projected 2 mm past the guide cannula to the central nucleus of the amygdala. The injectors were attached to 70 cm of calibrated polyethylene-20 tubing preloaded with drug solution. This cohort of rats had been extensively handled previously in the context of a food intake study, in which they received administration of a CRF₁ receptor antagonist subcutaneously and into the central nucleus of the amygdala in a Latin-square design. A washout period of 7 days was imposed before the present study with all subjects maintained on chow, during which animals were handled daily. Rats were randomly assigned to CRF vs. vehicle conditions balanced for previous diet history, which was statistically unrelated to performance in the defensive burying test. The CRF group (n=5) was infused bilaterally (30 pmol total dose) with a volume of 0.25 μL/side over 30 seconds using Hamilton microsyringes and 2 infusion pumps (Harvard Apparatus, Holliston, Mass.). The control group (n=5) received the same volume of PBS. Injectors were removed from guide cannulae 1 minute after the end of the infusions, and rats were returned to the home cage for 1 minute before being tested in the defensive burying test.

Nicotine Self-Administration

The apparatus and detailed procedures for both intravenous catheterization and self-administration of nicotine have been described previously [O'dell et al., Pharmacol. Biochem. Behay. (2007) 86:346-353]. Adult male Wistar rats (280 to 330 g) were first allowed to nose-poke for food and water in 23 hours sessions prior to and after recovery from surgical implantation of jugular catheters. Following acquisition of these operant responses, rats were allowed to self-administer nicotine (0.03 mg/kg/100 μl/1 s, free base, fixed ratio=1 lever-response, time out=20 seconds) under different paradigms.

Study A: Effect of MPZP in ShA Rats

Rats were allowed to acquire nicotine self-administration during daily 1 hour, “short-access” sessions (ShA, n=10) for at least 10 days. The CRF₁ antagonist MPZP (0, 5, 10, 20 mg/kg) was then administered using a Latin square design, with 1-2 intervening treatment-free days.

Study B: Effect of Nicotine Deprivation in ShA and LgA Rats

ShA (n=6) and LgA rats (n=7) were allowed to self-administer nicotine during daily sessions during at least 10 days. Then, they were submitted to 3 days of abstinence in their home cage followed by one session of nicotine self-administration to assess the magnitude of the nicotine deprivation effect. ShA and LgA rats were then left undisturbed in the vivarium for a 1 month period and used for Study C. Catheter patency was tested using an ultra short-acting barbiturate, Brevital (methohexital sodium, 10 mg/ml, 2 mg/rat), and only rats with a fully patent catheter were used.

Study C: Effect of MPZP on the Nicotine Deprivation Effect, in LgA Rats

After completion of Study B, rats were allowed to self-administer nicotine during daily 23 hours, “long-access” sessions (LgA, n=8) for at least 10 days. Then, they were allowed to respond on a lever for nicotine self-administration in 4 four-day cycles, each separated by three intervening days of abstinence in their home cage. MPZP was administered before the first session following each cycle of abstinence using a Latin square design.

Study D: Further Characterization of the Nicotine Deprivation Effect

After completion of Study C, rats were submitted to 9 successive cycles of nicotine self-administration periods and abstinence periods. The different durations of abstinence were tested in the following order 72 hours, 48 hours, 265 hours, 12 hours, 1201 hours. The order of testing was pseudo-randomized to avoid confounding effects due to the order of testing with those due to the duration of abstinence. After each abstinence period rats were allowed to self-administrate nicotine until they reached their pre-deprivation baseline (range 3-5 days). The 72 hours cycle period was repeated four times to analyze the reproducibility of the results (FIG. 7A).

Statistical Analysis

Results were analyzed with SPSS software using analysis of variance (ANOVA). In all cases a normality test and an equal variance test were performed before the ANOVA to ensure its validity. The following variables (dependent/non-dependent: 2 levels; sham/abstinence: 2 levels; duration of nicotine access: 2 levels; pharmacological treatments: 2 or 4 levels; active/inactive response: 2 levels) were used as between-subjects factor. Depending on the analysis, the condition (baseline/post-abstinence: 2 levels) and the time (number of self-administration sessions or number of microdialysis samples) were used as within-subjects factors. Post hoc Newman-Keuls tests and Pearson correlations were used when necessary. When assumptions of ANOVA were violated the non-parametric Kruskal-Wallis test was used, followed by the Welch's t-test. Data are shown as mean±S.E.M.

Attenuation of Escalated Cocaine Self-administration in Rats

### Materials and Methods

Animals

Thirty-two male Wistar rats (Charles River, Kingston, N.Y.), weighing 250-300 g at the start of the experiment, were group-housed (2-3 per cage) on a reverse light/dark cycle (lights off 0800 to 2000), in a climate-controlled vivarium. All behavioral testing occurred during the dark cycle. Food and water were freely available unless otherwise specified. All procedures met the guidelines of the National Institutes of Health Guide for the Care and Use of Laboratory Animals (The National Academies Press, 1996).

Drugs

Cocaine hydrochloride (National Institute on Drug Abuse, Rockville Md.) was dissolved in sterile physiological saline to 0.25 mg/0.1 ml/infusion. N-Butyl-N-ethyl-2,5,6-trimethyl-7-(2,4,6-trimethyl-phenyl)-7H-pyrrolol[2,3-d]pyrimidin-4-amine hydrochloride (antalarmin hydrochloride, Sigma, St. Louis Mo.) (below) was initially dissolved in 1 M HCl (a volume equal to 5% final volume), then suspended in a 0.5% (w/v) low-viscosity carboxymethylcellulose (Sigma) saline solution. This solution was back-titrated with 1 M NaOH to a pH value of about 4 and injected at volume of 5 ml/kg. MPZP was synthesized by Dr. Peter Wirsching (Department of Chemistry, The Scripps Research Institute). The methoxy substituents in the “top” MPZP alkyl unit confer increased hydrophilicity compared to the parent compound or antalarmin, yielding a CRF₁ antagonist with lipophilicity more typical of central nervous system-acting drug-like molecules (Zorrilla and Koob, 2004). MPZP was solubilized in 1 M HCl, diluted in hydroxypropyl β-cyclodextrin (20% w/v final concentration, Cavitron 82004, Cargill, Wayzata Minn.) saline solution, back-titrated with NaOH to a final pH value of 4.5, and injected at a volume of 2 ml/kg. FIG. 1 presents the CRF₁ antagonists under study.

Both MPZP and antalarmin (below) have high

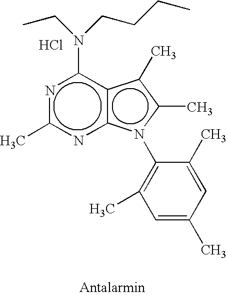

affinity for rat CRF₁ receptors (Table 1). Like antalarmin, MPZP is a selective CRF₁ antagonist. In vitro receptor autoradiography studies have shown that MPZP does not displace [¹²⁵I]-Tyr⁰-sauvagine binding from rat lateral septum or ventromedial hypothalamus (CRF₂-like binding) at a concentration (1 mM) that concurrently displaces the majority of [¹²⁵O]-Tyr⁰-sauvagine from cerebral cortex (CRF₁-like binding). Although the binding affinity of MPZP for CRF₁ receptors is slightly less potent than that of antalarmin, MPZP has lipophilicity 3.5 to 4 times lower than that of antalarmin and in a range more typical of central nervous system-acting therapeutics [compare cLogP and cLogD in Table 1; Zorrilla et al., Expert Opin. Investig. Drugs (2004) 13:799-828]. The molecular volume and polar surface area of MPZP also are consistent with an absorbable, blood-brain barrier-penetrating molecule [Kelder et al., Pharm. Res. (1999) 16:1514-1519; Zhao et al., J. Chem. Inf. Model (2007) 47:170-175; Fu et al., Pharmazie (2005) 60:354-358; Liu et al. Drug Metab. Dispos. (2004) 32:132-139].

Apparatus

Behavioral training occurred in operant conditioning chambers (Coulbourn Instruments, Allentown Pa.) housed in sound-attenuating cubicles. All chambers were equipped with two retractable levers, a dispenser for food pellets (P.J. Noyes Co., Lancaster N.H.) and a syringe pump (Model A, Razel Scientific Instruments, Stamford Conn.) delivering 0.1 ml of cocaine solution over 4 seconds via Tygon® tubing attached to liquid swivels (Model 375, Instech Labs, Plymouth Meeting Va.). A time-out (20 seconds) followed each infusion, during which a cue-light above the active lever was illuminated. At the start of a session, two levers were presented. Responding on the active lever resulted in reinforcement, whereas responding on the inactive lever resulted in no consequences but was recorded. Sessions were controlled and recorded by a personal computer with a custom interface and software.

Intravenous Surgery

Rats were implanted with an indwelling catheter into the right jugular vein under 1-3% isoflurane as described by Caine et al. [“Intravenous drug-self-administration techniques in animals”. In: Sahgal A, editor, Behavioural Neuroscience: a practical approach, vol 2, Oxford University Press, New York, (1993) pp., 117-143]. Catheters were flushed daily with 0.2 ml of sterile antibiotic solution containing Timentin (100 mg/ml; SmithKline Beecham Pharmaceuticals, Philadelphia Pa.) and heparin (30 USP units/ml). Catheter patency was checked by briefly aspirating blood from the catheter.

Self-Administration Procedure

Initially, rats were food-restricted (15 g/rat/day) and trained to press a lever for a food pellet (45 mg Formula A/I, Research Diets, New Brunswick N.J.) under a fixed-ratio (FR) 1 schedule in 30 min sessions, twice daily for a total of 5 days before intravenous catheterization. During this period, the length of time-out following reinforcement was gradually increased (1, 5, 10, 20 seconds). After the animal reached the 20 second time-out, food was available ad libitum for the remainder of the study. The rats then were implanted with intravenous catheters as described above.

After recovery from surgery, rats self-administered 0.25 mg/infusion (0.66 mg/kg/infusion) of cocaine in daily 1 hour sessions under an FR1 schedule for a maximum of 11 days. Following these baseline sessions, animals were separated into two groups, balanced for body weight and cocaine intake. The session length was kept to 1 hour for one group (short access, ShA, n=16) and was increased to 6 hours for the other group (long access, LgA, n=16; escalation period). Sessions in this escalation period lasted for 11 to 15 days before testing with CRF₁ receptor antagonists. Following the escalation period, the effects of antalarmin or MPZP on cocaine self-administration were tested in separate groups of ShA and LgA rats under an FR1 schedule. The antalarmin (6.25-25 mg/kg) pretreated animals were injected intraperitoneally 80 minutes before a test session. The MPZP (3.6-27.5 mg/kg) pretreated animals were injected subcutaneously 45 min before a test session. These doses and pretreatment time intervals were chosen based on previous experience with the anti-stress time course and potencies of these compounds [Zorrilla et al., Brain Res (2002) 952:188-99; Fekete et al., Society for Neuroscience (2003) Program No. 538.13. Abstract Viewer and Itinerary Planner, Washington, D.C.]. Doses of each drug were tested in a Latin square design. Test sessions were 1 hour long and separated by 1-2 treatment-free daily escalation sessions.

Data Analyses

Data were expressed as the first hour and total session cocaine intake (mg/kg). To analyze changes in cocaine intake during the escalation period, a mixed-design two-way analysis of variance (ANOVA) was used, with Access as a between-subjects factor and Session as a within-subjects factor. The effects of the CRF₁ receptor antagonists on cocaine intake were evaluated using separate mixed-design two-way ANOVAs, with Access as a between-subjects factor and Dose as a within-subjects factor. Previous research in our laboratory demonstrated that rats self-administer more drug during the first 10 minutes (loading phase) than in any other 10 minute period of a 1 hour session [Kitamura et al., Psychopharmacology (2007) 186:48-53; Wee et al. (2007) J. Pharmacol. Exp. Ther. (2007) 320:1134-1143]. Therefore, in addition to the effect of CRF₁ antagonists on cocaine intake of ShA and LgA rats, we further analyzed the effect of MPZP and antalarmin on the pattern of cocaine intake within a test session in LgA rats. Data for the LgA rats in each antagonist-treated group compared the loading phase intake (first 10 minutes) with the average for the time from 10-60 minutes and subjected to a two-way ANOVA. Linear trends analysis was used to determine whether CRF₁ antagonists exhibited log-linear dose-dependent effects on cocaine intake during the test sessions. Post hoc comparisons were performed using the Student-Neuman-Keuls test for cocaine intake during escalation, and Dunnett's tests were used to interpret CRF₁ antagonist dose effects during testing. The statistical packages used were Statview (SAS Institute, Cary N.C.), SPSS (Chicago Ill.), Instat, and Prism (GrapPad, San Diego Calif.).

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. A method for treating a host mammal that exhibits aversive signs and symptoms present during protracted abstinence or extended discontinuation syndromes as seen after cessation of compulsive activity, behaviors, or substance use that comprises the steps of
administering to a host mammal in need thereof a pharmaceutical composition containing an aversive sign and symptom lessening amount a compound of Formula I or a pharmaceutically acceptable salt thereof dissolved or dispersed in a physiologically acceptable diluent, and repeating the administration as needed, 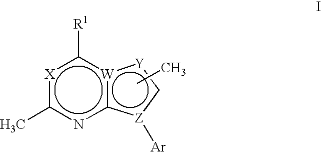
wherein
W and Z are independently N or C, and X and Y are independently N or CH, with the proviso that at least two and no more that three of W, X, Y and Z are N;
R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;
Ar—is 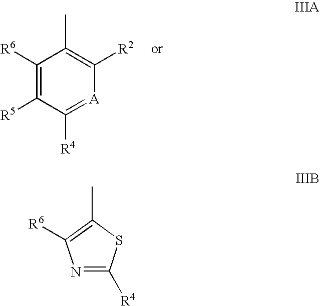
wherein
A is CH or N,
R² is selected from the group consisting of hydrido, methyl, methoxy, chloro and bromo,
R⁴ is selected from the group consisting of chloro, methyl, methoxy, dimethylamino and morpholinyl,
R⁵ is selected from the group consisting of hydrido, chloro and methyl, and
R⁶ is selected from the group consisting of hydrido, chloro, methyl and methoxy,
said compound exhibiting a calculated cLogD, pH 7 value of about 1.5 to about 4.5, using ACD/Labs Software v.8.14 for Solaris, a pKₐ value of about 4 to about 8.5, and a polar surface area of about 40 to about 70 Å².

2. A method for treating a host mammal that exhibits substance-related or substance-induced psychiatric disorders that include aversive signs and symptoms that comprises the steps of
administering to a host mammal in need thereof a pharmaceutical composition containing a substance-related or substance-induced psychiatric disorder aversive sign and symptom lessening amount a compound of Formula I or a pharmaceutically acceptable salt thereof dissolved or dispersed in a physiologically acceptable diluent, and repeating the administration as needed, 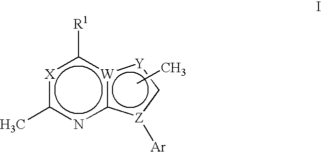
wherein
W and Z are independently N or C, and X and Y are independently N or CH, with the proviso that at least two and no more that three of W, X, Y and Z are N;
R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;
Ar—is 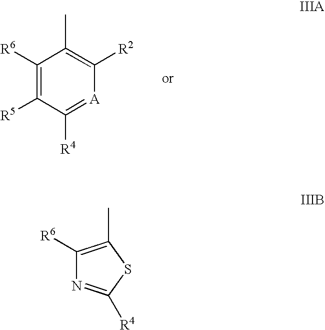
wherein
A is CH or N,
R² is selected from the group consisting of hydrido, methyl, methoxy, chloro and bromo,
R⁴ is selected from the group consisting of chloro, methyl, methoxy, dimethylamino and morpholinyl,
R⁵ is selected from the group consisting of hydrido, chloro and methyl, and
R⁶ is selected from the group consisting of hydrido, chloro, methyl and methoxy,
said compound exhibiting a calculated cLogD, pH 7 value of about 1.5 to about 4.5, using ACD/Labs Software v.8.14 for Solaris, a pKₐ value of about 4 to about 8.5, and a polar surface area of about 40 to about 70 Å².

3. A method for treating a host mammal to inhibit relapse of compulsive use or behavioral disorders that comprises the steps of
administering to a host mammal in need thereof a pharmaceutical composition containing a compulsive use or behavioral disorders relapse inhibiting amount a compound of Formula I or a pharmaceutically acceptable salt thereof dissolved or dispersed in a physiologically acceptable diluent, and repeating the administration as needed, 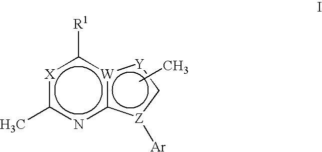
wherein
W and Z are independently N or C, and X and Y are independently N or CH, with the proviso that at least two and no more that three of W, X, Y and Z are N;
R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;
Ar—is 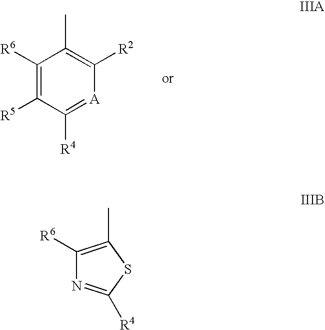
wherein
A is CH or N,
R² is selected from the group consisting of hydrido, methyl, methoxy, chloro and bromo,
R⁴ is selected from the group consisting of chloro, methyl, methoxy, dimethylamino and morpholinyl,
R⁵ is selected from the group consisting of hydrido, chloro and methyl, and
R⁶ is selected from the group consisting of hydrido, chloro, methyl and methoxy,
said compound exhibiting a calculated cLogD, pH 7 value of about 1.5 to about 4.5, using ACD/Labs Software v.8.14 for Solaris, a pKₐ value of about 4 to about 8.5, and a polar surface area of about 40 to about 70 Å².

4. A method for preventing a host mammal from exhibiting aversive signs and symptoms present during protracted abstinence or extended discontinuation syndromes as seen after cessation of compulsive activity, behaviors, or substance use that comprises the steps of
administering to a host mammal in need thereof a pharmaceutical composition containing an aversive sign and symptom present during protracted abstinence or extended discontinuation syndrome-preventing amount a compound of Formula I or a pharmaceutically acceptable salt thereof dissolved or dispersed in a physiologically acceptable diluent, and repeating the administration as needed, 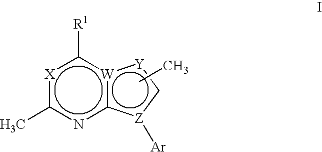
wherein
W and Z are independently N or C, and X and Y are independently N or CH, with the proviso that at least two and no more that three of W, X, Y and Z are N;
R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;
Ar—is 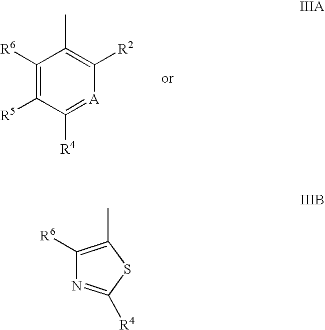
wherein
A is CH or N,
R² is selected from the group consisting of hydrido, methyl, methoxy, chloro and bromo,
R⁴ is selected from the group consisting of chloro, methyl, methoxy, dimethylamino and morpholinyl,
R⁵ is selected from the group consisting of hydrido, chloro and methyl, and
R⁶ is selected from the group consisting of hydrido, chloro, methyl and methoxy,
said compound exhibiting a calculated cLogD, pH 7 value of about 1.5 to about 4.5, using ACD/Labs Software v.8.14 for Solaris, a pKₐ value of about 4 to about 8.5, and a polar surface area of about 40 to about 70 | ².

5. The method according to claim 1, wherein said compound of Formula I is comprised of a core bonded to R¹ and Ar substituents in which the core corresponds to structural Formula II with bond lines for substituents R¹ and Ar 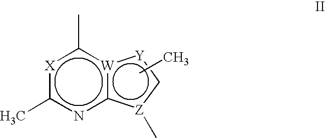
wherein said core contains 3 or 4 nitrogen atoms in the rings, including the nitrogen atom shown in Formula II.

6. The method according to claim 5, wherein said core corresponds in structure to one of Formulas IIA, IIB, IIC or IID

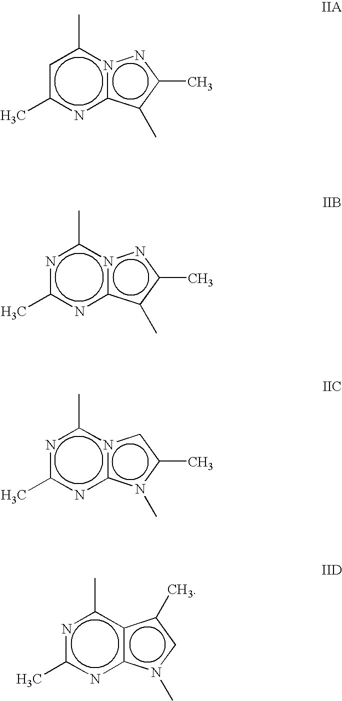

7. The method according to claim 1, wherein Ar corresponds to Formula IIIA.

8. The method according to claim 7, wherein A in Formula IIIA is CH.

9. The method according to claim 5, wherein said each of R⁷ and R⁸ groups is the same.

10. The method according to claim 9, wherein said each of R⁷ and R⁸ groups includes a methoxy or hydroxy group.

11. The method according to claim 1, wherein said aversive signs and symptoms are present during protracted abstinence or extended discontinuation syndromes after cessation of compulsive activity.

12. The method according to claim 1, wherein said aversive signs and symptoms are present during protracted abstinence or extended discontinuation syndromes after cessation of substance use.

13. The method according to claim 12, wherein said substance is alcohol.

14. The method according to claim 12, wherein said substance is nicotine.

15. The method according to claim 12, wherein said substance is cocaine.

16. The method according to claim 1, wherein said calculated cLogD, pH 7 value is about 2.0 to about 3.5.

17. The method according to claim 1, wherein said compound of Formula I corresponds to the formula

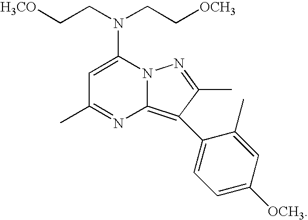

18. A pharmaceutical composition that contains an effective amount of a compound of Formula I or a pharmaceutically acceptable salt thereof dissolved or dispersed in a physiologically acceptable diluent, 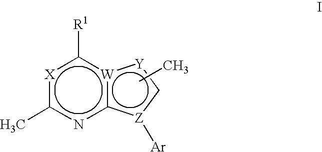
wherein
W and Z are independently N or C, and X and Y are independently N or CH, with the proviso that at least two and no more that three of W, X, Y and Z are N, such that said compound of Formula I is comprised of a core bonded to R¹ and Ar substituents in which the core corresponds to structural Formula II with bond lines for substituents R¹ and Ar 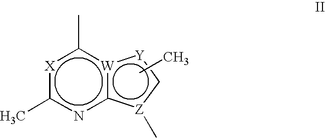
wherein said core contains 3 or 4 nitrogen atoms in the rings, including the nitrogen atom shown in Formula II and core corresponds in structure to one of Formulas IIA, IIB, IIC or IID 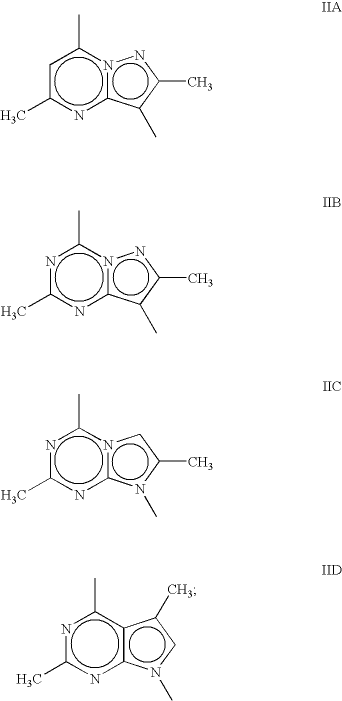
R¹ is NR⁷R⁸ where each of R⁷ and R⁸ is independently selected from the group consisting of 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;
Ar—is 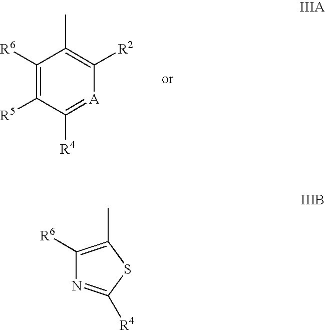
wherein
A is CH or N,
R² is selected from the group consisting of hydrido, methyl, methoxy, chloro and bromo,
R⁴ is selected from the group consisting of chloro, methyl, methoxy, dimethylamino and morpholinyl,
R⁵ is selected from the group consisting of hydrido, chloro and methyl, and
R⁶ is selected from the group consisting of hydrido, chloro, methyl and methoxy,
said compound exhibiting a calculated cLogD, pH 7 value of about 1.5 to about 4.5, using ACD/Labs Software v.8.14 for Solaris, a pKₐ value of about 4 to about 8.5, and a polar surface area of about 40 to about 70 Å².

19. The pharmaceutical composition according to claim 18, wherein a compound of Formula I or a pharmaceutically acceptable salt thereof dissolved or dispersed in a physiologically acceptable diluent is, 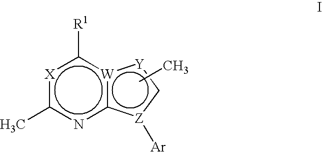
wherein
W and Z are independently N or C, and X and Y are independently N or CH, with the proviso that at least two and no more that three of W, X, Y and Z are N;
R¹ is NR⁷R⁸ where each of R⁷ and R⁸ is independently a straight, branched or cyclic substituent that is selected from the group consisting of C₁-C₄ alkyl or C₁-C₄ alkenyl, methoxy-C₁-C₃ alkyl or C₁-C₃ alkenyl, mono-or dihydroxy-C₁-C₃ alkyl or C₁-C₃ alkenyl, N-methylamino-C₁-C₃ alkyl or C₁-C₃ alkenyl, 2- or 3-tetrahydrofuryl, and 2- or 3-tetrahydrofurfuryl, or NR⁷R⁸ together form a 5- or 6-membered ring containing zero or one oxygen atom in the ring, which ring is unsubstituted or substituted with a hydroxyl group, a hydroxymethyl group or a hydroxyethyl group;
Ar— is 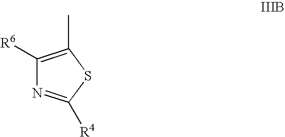
wherein
R⁴ is selected from the group consisting of chloro, methyl, methoxy, dimethylamino and morpholinyl, and
R⁶ is selected from the group consisting of hydrido, chloro, methyl and methoxy,
said compound exhibiting a calculated cLogD, pH 7 value of about 1.5 to about 4.5, using ACD/Labs Software v.8.14 for Solaris, a pKₐ value of about 4 to about 8.5, and a polar surface area of about 40 to about 70 Å².

20. The pharmaceutical composition according to claim 18, wherein said compound of Formula I corresponds to the formula [chemical structure]
